(19) United States
(12) Patent Application Publication
(10) Pub. No.: US 2010/0004452 A1
(43) Pub. Date: Jan. 7, 2010

(54) 5-QUINOLINONE DERIVATIVES AS 5HT1A RECEPTOR MODULATORS FOR TREATING SEXUAL DYSFUNCTION, COGNITION IMPAIRMENT, PSYCHOTIC DISORDERS, ANXIETY, DEPRESSION, ETC.

(76) Inventors: Barbara Bertani, Verona (IT); Steven Mark Bromidge, Essex (GB); Massimo Gianotti, Verona (IT); Alessandra Pasquarello, Verona (IT); Valeria Zucchelli, Verona (IT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/442,148

(22) PCT Filed: Sep. 24, 2007
(86) PCT No.: PCT/EP2007/060082

§ 371 Date: Mar. 20, 2009

(30) Foreign Application Priority Data
Sep. 26, 2006 (GB) 0618949.2
Jun. 22, 2007 (GB) 0712147.8

(51) Int. Cl. C07D 401/14

(52) U.S. Cl. 544/363

(57) ABSTRACT

Disclosed are compounds of formula (I)

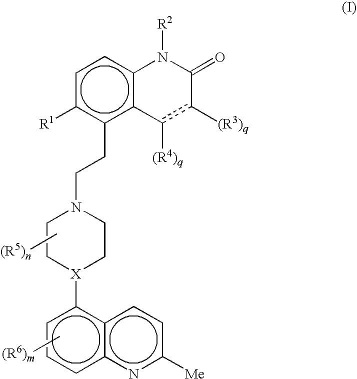

wherein R¹ is C₁₋₆alkyl, halo or haloC₁₋₆alkyl; R² is hydrogen or C₁₋₆alkyl; [symbol] is a single or double bond; each R³ and R⁴, which may be the same or different, are hydrogen, C₁₋₆alkyl or haloC₁₋₆alkyl; wherein i) when [symbol] is a double bond p and q are 1, and ii) when [symbol] is a single bond, p and q are 2, and one of R³ and one of R⁴, together with their interconnecting atoms, forms a cyclopropane ring which may be substituted by one or two halo or methyl groups, which groups may be the same or different; X is CH or N; when present each R⁵, which may be the same or different, is C₁₋₆alkyl or halo; or two R⁵ groups may join to form a bridge, which bridge contains one or two atoms; n is 0, 1, 2 or 3; when present each R⁶ which may be the same or different, is C₁₋₆alkyl or halo; and m is 0, 1, 2 or 3.

This invention relates to novel quinolinone derivatives. The invention also relates to the use of the derivatives in treating diseases and conditions mediated by antagonism of the 5-HT_{1A} receptor. In addition, the invention relates to compositions containing the derivatives and processes for their preparation.

According to a first aspect, the invention provides a compound of formula (I)

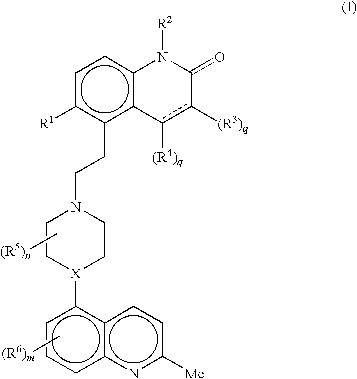

wherein
R¹ is C₁₋₆alkyl, halo or haloC₁₋₆alkyl;
R² is hydrogen or C₁₋₆alkyl;
[symbol] is a single or double bond; wherein when [symbol] is a double bond p and q are 1; and when [symbol] is a single bond, p and q are 2;
each R³ and R⁴, which may be the same or different, are hydrogen, C₁₋₆alkyl or haloC₁₋₆alkyl; or when [symbol] is a single bond, one of R³ and one of R⁴, together with their interconnecting atoms, forms a cyclopropane ring which may be substituted by one or two halo or methyl groups, which groups may be the same or different;
X is CH or N;
when present each R⁵, which may be the same or different, is C₁₋₆alkyl or halo; or two R⁵ groups may join to form a bridge, which bridge contains one or two atoms;
n is 0, 1, 2 or 3;
when present each R⁶ which may be the same or different, is C₁₋₆alkyl or halo; and m is 0, 1, 2 or 3.

Unless otherwise indicated, any alkyl group is straight or branched regardless of whether it forms part of another group, for example, alkoxy, haloalkyl and haloalkoxy.

As used herein, a haloalkyl group means an alkyl group substituted by one or more halogen atoms. A haloalkoxy group should be similarly construed.

Halo means fluoro, chloro, bromo or iodo.

For the avoidance of doubt, when present, the one, two or three R⁵ groups may be attached to the piperazine or piperidine ring at any appropriate position.

For the avoidance of doubt, when present, the one, two or three R⁶ groups may be attached to the quinoline ring at any appropriate position.

In an embodiment,
R¹ is C₁₋₆alkyl, halo or haloC₁₋₆alkyl;
R² is hydrogen or C₁₋₆alkyl;
[symbol] is a single or double bond;
each R³ and R⁴, which may be the same or different, are hydrogen, C₁₋₆alkyl or haloC₁₋₆alkyl; wherein
   i) when [symbol] is a double bond p and q are 1, and
   ii) when [symbol] is a single bond, p and q are 2, and one of R³ and one of R⁴, together with their interconnecting atoms, forms a cyclopropane ring which may be substituted by one or two halo or methyl groups, which groups may be the same or different;
X is CH or N;
when present each R⁵, which may be the same or different, is C₁₋₆alkyl or halo; or two R⁵ groups may join to form a bridge, which bridge contains one or two atoms;
n is 0, 1, 2 or 3;
when present each R⁶ which may be the same or different, is C₁₋₆alkyl or halo; and
m is 0, 1, 2 or 3.

In an embodiment, R¹ is C₁₋₆alkyl. In a further embodiment, R¹ is C₁₋₃alkyl. In a still further embodiment R¹ is methyl.

In an embodiment R² is hydrogen or C₁₋₆alkyl. In a further embodiment R² is hydrogen or C₁₋₃alkyl. In a still further embodiment R² is hydrogen or methyl. In a still further embodiment R² is hydrogen.

In an embodiment [symbol] is a single bond and each R³ and each R⁴ are hydrogen.

In an embodiment X is N.

In an embodiment n is 0, 1 or 2. In a further embodiment n is 0 or 1. In a still further embodiment n is 0.

In an embodiment, when present, each R⁵ is C₁₋₆alkyl. In a further embodiment, when present, each R⁵ is C₁₋₃alkyl. In a still further embodiment, when present, each R⁵ is methyl.

In an embodiment, when present, each R⁵ is attached to the piperazine or piperidine ring at one or both of the carbon atoms next to the nitrogen attached to the ethylene chain in formula (I).

In an embodiment, when two R⁵ groups join to form a bridge, the bridge contains two carbon atoms and the bridge is attached to non-adjacent carbon atoms in the piperazine or piperidine ring.

In an embodiment m is 0 or 1. In a further embodiment m is 0.

In an embodiment, when present, R⁶ is attached to the 7-position of the quinoline ring.

In an embodiment,

R¹ is C₁₋₆alkyl;
R² is hydrogen; [symbol]
is a single bond and each R³ and R⁴, are hydrogen;

### X is N;

n is 0, 1 or 2;
when present each R⁵ is C₁₋₆alkyl;
m is 0 or 1; and
when present R⁶ is C₁₋₆alkyl or halo.

In an embodiment, the compounds of formula (I) are selected from the list consisting of:
6-methyl-5-{2-[4-(2-methylquinolin-5-yl)piperazin-1-yl]ethyl}-3,4-dihydroquinolin-2(1H)-one (Compound 1);
5-{2-[(2S)-4-(7-fluoro-2-methylquinolin-5-yl)-2-methylpiperazin-1-yl]ethyl}-6-methyl-3,4-dihydroquinolin-2(1H)-one (Compound 2);
1,6-dimethyl-5-{2-[4-(2-methylquinolin-5-yl)piperazin-1-yl]ethyl}-3,4-dihydroquinolin-2(1H)-one (Compound 7);
6-methyl-5-{2-[4-(2-methyl-5-quinolinyl)-1-piperazinyl]ethyl}-2(1H)-quinolinone (Compound 14);
5-{2-[4-(7-fluoro-2-methyl-5-quinolinyl)-1-piperazinyl]ethyl}-6-methyl-2(1H)-quinolinone (Compound 15);
6-methyl-5-{2-[4-(2-methyl-5-quinolinyl)-1-piperidinyl]ethyl}-2(1H)-quinolinone (Compound 16);
5-{2-[4-(7-fluoro-2-methyl-5-quinolinyl)-1-piperazinyl]ethyl}1,6-dimethyl-2(1H)-quinolinone (Compound 19);
1,6-dimethyl-5-{2-[(2S)-2-methyl-4-(2-methyl-5-quinolinyl)-1-piperazinyl]ethyl}2(1H)-quinolinone (Compound 21);
5-{2-[(2S)-4-(7-fluoro-2-methyl-5-quinolinyl)-2-methyl-1-piperazinyl]ethyl}-1,6-dimethyl-2(1H)-quinolinone (Compound 23); and
3,6-dimethyl-7-{2-[(2S)-2-methyl-4-(2-methyl-5-quinolinyl)-1-piperazinyl]ethyl}-1,1a,3,7b-tetrahydro-2H-cyclopropa[c]quinolin-2-one (Compound 33);

In a further embodiment, the compound of formula (I) is 6-methyl-5-{2-[4-(2-methylquinolin-5-yl)piperazin-1-yl]ethyl}-3,4-dihydroquinolin-2(1H)-one (Compound 1).

The compounds defined in the first aspect may form pharmaceutically or veterinarily acceptable salts. Therefore according to a further aspect, the invention provides a pharmaceutically acceptable salt of a compound defined in the first aspect and embodiments thereof.

The compounds defined in the first aspect contain a basic centre and may form non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497.

It will be appreciated by those skilled in the art that certain protected derivatives of compounds defined in the first aspect, which may be made prior to a final deprotection stage, may not possess pharmacological activity as such, but may, in certain instances, be administered orally or parenterally and thereafter metabolised in the body to form compounds defined in the first aspect which are pharmacologically active. Such derivatives may therefore be described as “prodrugs”. All protected derivatives and prodrugs of compounds defined in the first aspect are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within the compounds defined in the first aspect.

The compounds defined in the first aspect or their pharmaceutically acceptable salts may exist in solvated or hydrated form.

The compounds defined in the first aspect, their pharmaceutically acceptable salts or solvates/hydrates of the compounds or salts, may exist in one or more polymorphic form.

Therefore, in a further aspect, the invention provides a pharmaceutically acceptable salt, solvate or prodrug of a compound defined in the first aspect.

Hereinafter, compounds defined in the first aspect, their pharmaceutically acceptable salts, solvates and prodrugs are referred to as “compounds of the invention”.

The compounds of the invention may possess one or more chiral centres and so exist in a number of stereoisomeric forms. All stereoisomers and mixtures thereof are included in the scope of the present invention. Racemic compounds may either be separated using preparative HPLC and a column with a chiral stationary phase or resolved to yield individual enantiomers utilising methods known to those skilled in the art. In addition, chiral intermediate compounds may be resolved and used to prepare chiral compounds of the invention.

The compounds of the invention may exist in one or more tautomeric forms. All tautomers and mixtures thereof are included in the scope of the present invention. For example, a claim to 2-hydroxyquinolinyl would also cover its tautomeric form, α-quinolinonyl.

Diastereoisomers of compounds of the invention may be obtained according to methods well known in the literature, for example by preparative HPLC or by chromatographic purifications. Racemic compounds may either be separated using preparative HPLC and a column with a chiral stationary phase or resolved to yield individual enantiomers utilising methods known to those skilled in the art. In addition, chiral intermediate compounds may be resolved and used to prepare chiral compounds of the invention.

The invention also includes all suitable isotopic variations of a compound of the invention. An isotopic variation of a compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorus, sulphur, fluorine and chlorine such as ²H, ³H, ¹¹C, ¹³C, ¹⁴C, ¹⁵N, ¹⁷O, ¹⁸O, ³¹P, ³²P, ³⁵S, ¹⁸F and ³⁶Cl, respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

Compounds of the invention may be prepared in a variety of ways. In the following reaction schemes and hereafter, unless otherwise stated R¹ to R⁶, X, n, m, p and q are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic) etc. . . . (IVa), (IVb), (IVc) etc.

Compounds of general formula (I) may be prepared by reacting compounds of formula (II) with compounds of formula (III) as shown in reaction scheme 1. Typical reaction conditions comprise stirring (II) and (III) in a suitable solvent (such 1,2-dichloroethane) at room temperature for 30 minutes, followed by treatment with a reducing agent such as sodium triacetoxyborohydride.

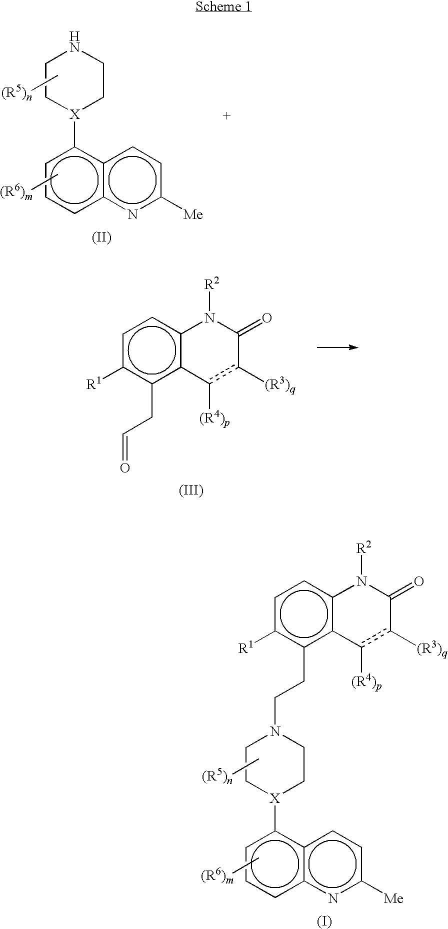

Compounds of formula (IIIa), i.e. compounds of general formula (III) where R¹ and R² are alkyl, [symbol] is a double bond and R³ and R⁴ are hydrogen, may be prepared according to reaction scheme 2. In a first step, compounds of formula (IV) are converted to the N-oxide (V) under conditions known in the art, such as treatment with 3-chloroperbenzoic acid. Treatment of compounds of formula (V) with trifluoroactic anhydride gives the quinolinone (VI). N-alkylation with sodium hydride and iodomethane gives (VII). Treatment of compounds of formula (VI) with allytributylstannane under palladium catalysis gives compounds of formula (VIII). Typical conditions for this step comprise treatment with palladium (0) tetrakistriphenylphosphine and lithium chloride in dimethylformamide at elevated temperatures, such as 100 degC. Finally, treatment of compounds of formula (VIII) with osmium tetroxide and sodium periodate under conditions known to the skilled chemist gives compounds of formula (IIIa).

Compounds of general formula (IV) are either commercially available or may be prepared by procedures known to the skilled person.

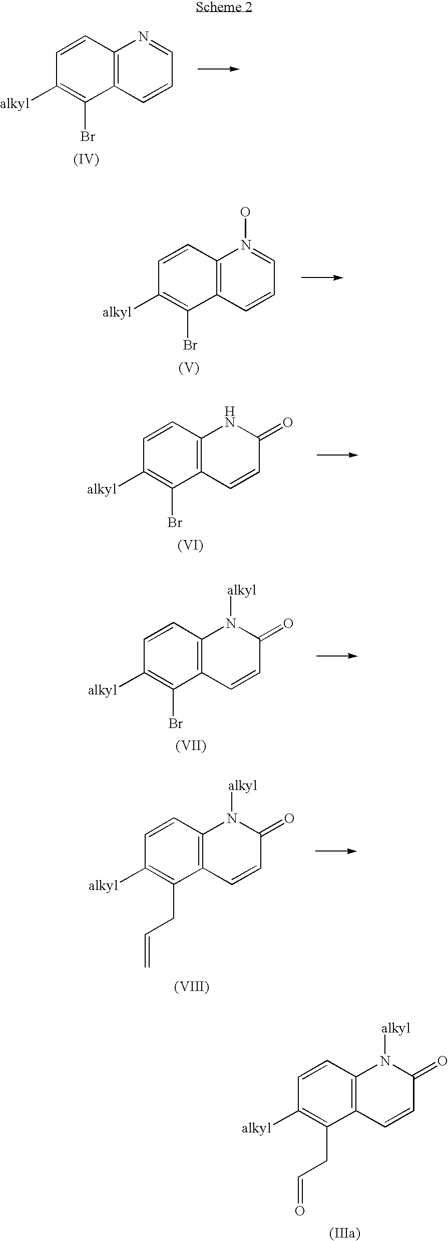

Compounds of formula (IIIb), i.e. compounds of general formula (III) where R¹ is alkyl, [symbol] is a single bond and each R³ and each R⁴ are hydrogen, may be prepared according to reaction scheme 3 by treatment of compounds formula (IX) with osmium tetroxide and sodium periodate under conditions known to the skilled chemist. Compounds of formula (IX) may be prepared using procedures similar to those described in WO2006/024517, Description 72.

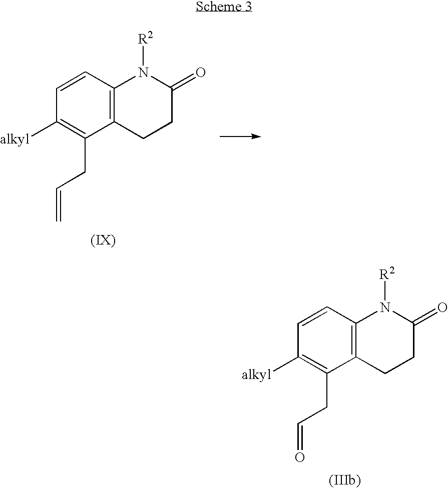

Compounds of formula (IIIc), i.e. compounds of general formula (III) where R¹ is alkyl, R² is hydrogen, [symbol] is a double bond and R³ and R⁴ are hydrogen, may be prepared according to reaction scheme 4. Firstly, compounds of formula (VI) (see reaction scheme 2) are reacted under palladium catalysis with 2-(ethenyloxy)-N,N-dimethylethanamine to give compounds of formula (X). Typical reaction conditions comprise treatment with palladium (II) acetate, triphenylphosphine and triethylamine at elevated temperature such as 100 degC. Treating (X) under acidic conditions (typically dilute aqueous sulphuric acid at room temperature) gives compounds of formula (IIIc).

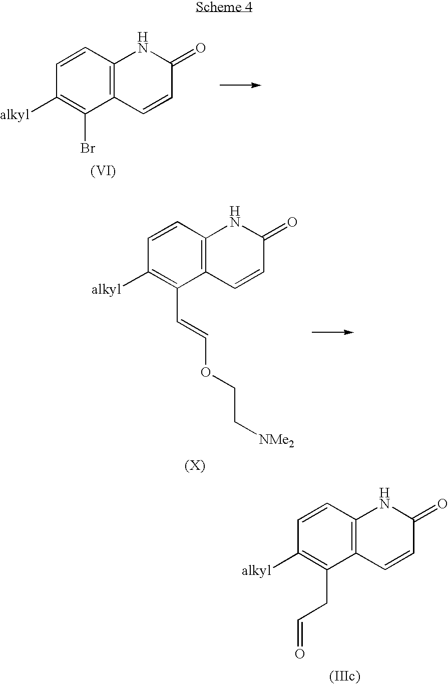

Compounds of formula (IIId), i.e. compounds of general formula (III) where R¹ is halo, R² is alkyl, [symbol] is a double bond and R³ and R⁴ are hydrogen, may be prepared according to reaction scheme 5. Firstly, compounds of formula (XII) may be prepared by reacting compounds of formula (X) with pivaloyl chloride, typically at ice-bath temperature in the presence of triethylamine. Treatment of (XII) with a strong base, such as n-butyllithium, followed by addition of dimethylformamide gives compounds of formula (XIII). Treatment with (carbethoxymethylene)triphenyl-phosphorane in a suitable solvent (such as toluene) and room temperature gives compounds of formula (XIV), which may be cyclised under acidic conditions to give compounds of formula (XV). Formation of the triflate, followed by reaction with allyltributylstannane under similar conditions to those described in reaction scheme 2, gives compounds of formula (XVIII). Reaction with osmium tetroxide and sodium periodate under standard conditions gives compounds of formula (IIId).

Compounds of general formula (XI) are either commercially available or may be prepared by procedures known to the skilled person.

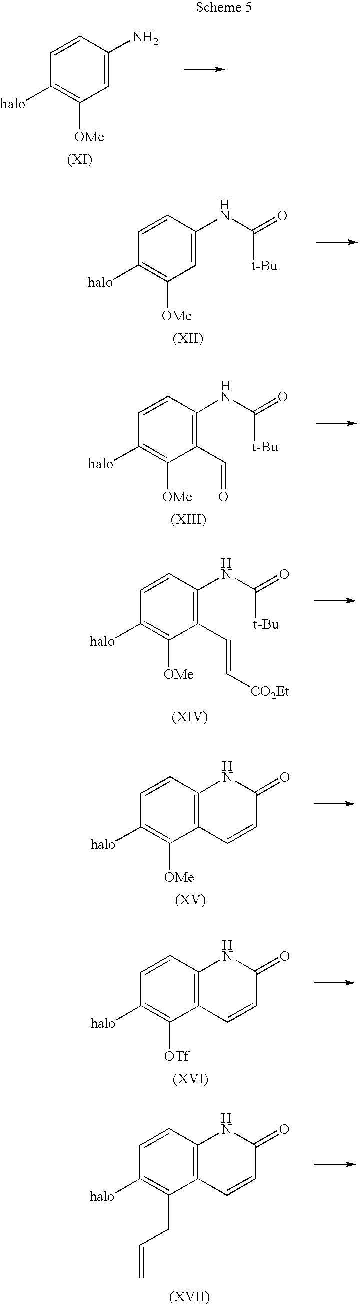

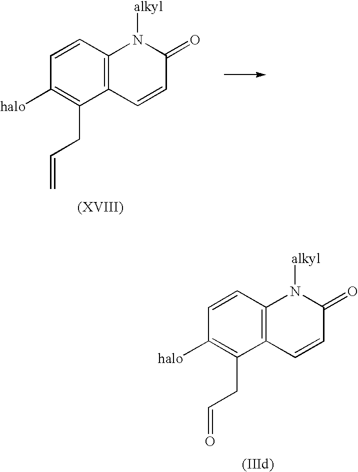

Compounds of formula (IIIe), i.e. compounds of general formula (III) where R¹ and R² are alkyl, [symbol] is a single bond and one of R³ and one of R⁴ together with their interconnecting atoms form a cyclopropane ring, may be prepared according to reaction scheme 6. Compounds of formula (XIX) may be prepared from compounds of formula (VII) (see reaction section 2) by adding (VII) to a base (such as sodium hydride) in a solution of trimethylsulfonium iodide followed by heating at elevated temperature. Compounds of formula (IIIe) may be obtained from (XIX) using methods described for reaction scheme 2.

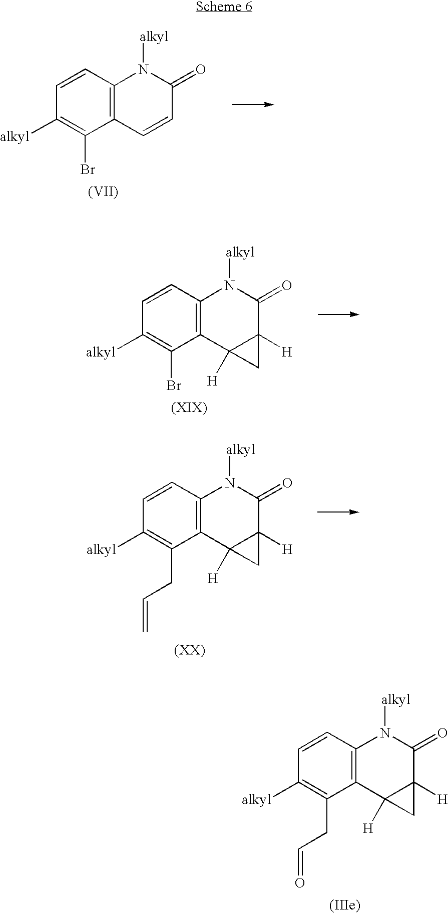

Compounds of formula (IIa), i.e. compounds of general formula (II) where X is N, may be prepared according to reaction scheme 7. Typically compounds of formula (XXI) and (XXII) are reacted in a suitable solvent (such as toluene) in the presence of a palladium catalyst (such as palladium (II) acetate), a base (such as caesium carbonate) and BINAP at elevated temperature.

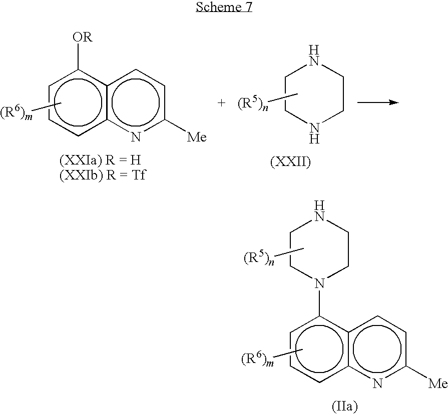

Compounds of general formula (XXIa/b) are either commercially available, known in the literature or may be prepared by procedures known to the skilled person.

Compounds of formula (IIb) may be prepared according to reaction scheme 8 by reacting compounds of formula (XXIb) with compounds of formula (XXIII) in a suitable solvent (such as DMF) in the presence of a palladium catalyst and a base (such as potassium carbonate) at elevated temperature, followed by reduction of double bond and removal of the butoxycarbonyl (BOC) protecting group under standard conditions (see WO2004/046124, Descriptions 16 and 18)

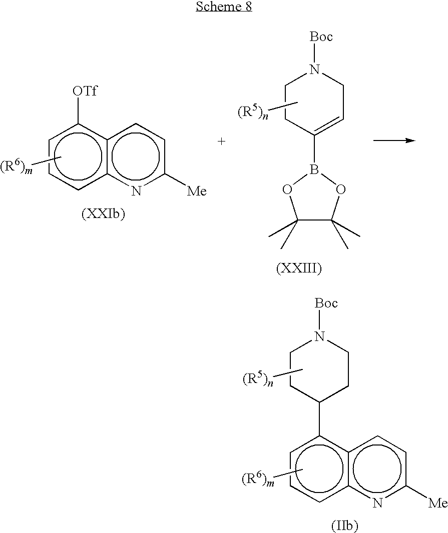

The compounds of the invention are effective antagonists of the 5-HT_{1A} receptor. In addition, some of the compounds of the invention are effective inhibitors of serotonin reuptake. In addition, the compounds of the invention are selective for the 5-HT_{1A} receptor over the 5-HT_{1B} receptor, i.e. the compounds are better antagonists of the 5-HT_{1A} receptor than they are antagonists of the 5-HT_{1B} receptor.

Therefore according to a further aspect, the invention provides a compound of the invention for use as a medicament, suitably a human medicament.

According to a further aspect, the invention provides the use of a compound of the invention in the manufacture of a medicament for treating or preventing a sexual dysfunction.

In an embodiment the sexual dysfunction is selected from the list consisting of: Sexual Desire Disorders (including Hypoactive Sexual Desire Disorder (302.71) and Sexual Aversion Disorder (302.79)); sexual arousal disorders (including Female Sexual Arousal Disorder (302.72) and Male Erectile Disorder (302.72)); orgasmic disorders (including Female Orgasmic Disorder (302.73), Male Orgasmic Disorder (302.74) and Premature Ejaculation (302.75)); sexual pain disorder (including Dyspareunia (302.76) and Vaginismus (306.51)); Sexual Dysfunction Not Otherwise Specified (302.70); paraphilias (including Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9)); gender identity disorders (including Gender Identity Disorder in Children (302.6) and Gender Identity Disorder in Adolescents or Adults (302.85)); and Sexual Disorder Not Otherwise Specified (302.9).

In a further embodiment, the sexual dysfunction is premature ejaculation.

According to a further aspect, the invention provides the use of a compound of the invention in the manufacture of a medicament for enhancing cognition including the treatment of cognition impairment associated with disease.

In an embodiment, the term “cognitive impairment” includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

In an embodiment, the disease associated with the cognition impairment is selected from the list: schizophrenia, bipolar disorder, depression, other psychiatric disorders and psychotic conditions associated with cognitive impairment, e.g. Alzheimer's disease.

In addition to the treatment of sexual dysfunction and for enhancing cognition including the treatment of cognition impairment associated with disease, the compounds of the invention may treat diseases or conditions selected from the list consisting of: [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:

i) Psychotic disorders for example Schizophrenia (including the subtypes Paranoid Type (295.30), Disorganised Type (295.10), Catatonic Type (295.20), Undifferentiated Type (295.90) and Residual Type (295.60)); Schizophreniform Disorder (295.40); Schizoaffective Disorder (295.70) (including the subtypes Bipolar Type and Depressive Type); Delusional Disorder (297.1) (including the subtypes Erotomanic Type, Grandiose Type, Jealous Type, Persecutory Type, Somatic Type, Mixed Type and Unspecified Type); Brief Psychotic Disorder (298.8); Shared Psychotic Disorder (297.3); Psychotic Disorder due to a General Medical Condition (including the subtypes with Delusions and with Hallucinations); Substance-Induced Psychotic Disorder (including the subtypes with Delusions (293.81) and with Hallucinations (293.82)); and Psychotic Disorder Not Otherwise Specified (298.9).
ii) Depression and mood disorders for example Depressive Episodes (including Major Depressive Episode, Manic Episode, Mixed Episode and Hypomanic Episode); Depressive Disorders (including Major Depressive Disorder, Dysthymic Disorder (300.4), Depressive Disorder Not Otherwise Specified (311)); Bipolar Disorders (including Bipolar I Disorder, Bipolar II Disorder (i.e. Recurrent Major Depressive Episodes with Hypomanic Episodes) (296.89), Cyclothymic Disorder (301.13) and Bipolar Disorder Not Otherwise Specified (296.80)); Other Mood Disorders (including Mood Disorder due to a General Medical Condition (293.83) which includes the subtypes With Depressive Features, With Major Depressive-like Episode, With Manic Features and With Mixed Features); Substance-Induced Mood Disorder (including the subtypes With Depressive Features, With Manic Features and With Mixed Features); and Mood Disorder Not Otherwise Specified (296.90).
iii) Anxiety disorders for example Social Anxiety Disorder; Panic Attack; Agoraphobia, Panic Disorder; Agoraphobia Without History of Panic Disorder (300.22); Specific Phobia (300.29) (including the subtypes Animal Type, Natural Environment Type, Blood-Injection-Injury Type, Situational Type and Other Type); Social Phobia (300.23); Obsessive-Compulsive Disorder (300.3); Posttraumatic Stress Disorder (309.81); Acute Stress Disorder (308.3); Generalized Anxiety Disorder (300.02); Anxiety Disorder Due to a General Medical Condition (293.84); Substance-Induced Anxiety Disorder; and Anxiety Disorder Not Otherwise Specified (300.00).
iv) Substance-related disorders for example Substance Use Disorders (including Substance Dependence, Substance Craving and Substance Abuse); Substance-Induced Disorders (including Substance Intoxication, Substance Withdrawal, Substance-Induced Delirium, Substance-Induced Persisting Dementia, Substance-Induced Persisting Amnestic Disorder, Substance-induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders (including Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9)); Amphetamine (or Amphetamine-Like)-Related Disorders (for example Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-induced Anxiety Disorder, Amphetamine-induced Sexual Dysfunction, Amphetamine-Induced Sleep Disorder and Amphetamine-Related Disorder Not Otherwise Specified (292.9)); Caffeine Related Disorders (including Caffeine Intoxication (305.90), Caffeine-Induced Anxiety Disorder, Caffeine-Induced Sleep Disorder and Caffeine-Related Disorder Not Otherwise Specified (292.9)); Cannabis-Related Disorders (including Cannabis Dependence (304.30), Cannabis Abuse (305.20), Cannabis Intoxication (292.89), Cannabis Intoxication Delirium, Cannabis-induced Psychotic Disorder, Cannabis-Induced Anxiety Disorder and Cannabis-Related Disorder Not Otherwise Specified (292.9)); Cocaine-Related Disorders (including Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9)); Hallucinogen-Related Disorders (including Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-induced Anxiety Disorder and Hallucinogen-Related Disorder Not Otherwise Specified (292.9)); Inhalant-Related Disorders (including Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9)); Nicotine-Related Disorders (including Nicotine Dependence (305.1), Nicotine Withdrawal (292.0) and Nicotine-Related Disorder Not Otherwise Specified (292.9)); Opioid-Related Disorders (including Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9)); Phencyclidine (or Phencyclidine-Like)-Related Disorders (including Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9)); Sedative-, Hypnotic-, or Anxiolytic-Related Disorders (including Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9)); Polysubstance-Related Disorder (including Polysubstance Dependence (304.80)); and Other (or Unknown) Substance-Related Disorders (including Anabolic Steroids, Nitrate Inhalants and Nitrous Oxide).

v) Sleep disorders for example primary sleep disorders such as Dyssomnias (including Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47)); primary sleep disorders such as Parasomnias (including Nightmare Disorder (307.47), Sleep Terror Disorder (307.46), Sleepwalking Disorder (307.46) and Parasomnia Not Otherwise Specified (307.47)); Sleep Disorders Related to Another Mental Disorder (including Insomnia Related to Another Mental Disorder (307.42) and Hypersomnia Related to Another Mental Disorder (307.44)); Sleep Disorder Due to a General Medical Condition; and Substance-Induced Sleep Disorder (including the subtypes Insomnia Type, Hypersomnia Type, Parasomnia Type and Mixed Type).

vi) Eating disorders such as Anorexia Nervosa (307.1) (including the subtypes Restricting Type and Binge-Eating/Purging Type); Bulimia Nervosa (307.51) (including the subtypes Purging Type and Nonpurging Type); Obesity; Compulsive Eating Disorder; Binge Eating Disorder; and Eating Disorder Not Otherwise Specified (307.50).

vii) Autism Spectrum Disorders including Autistic Disorder (299.00), Asperger's Disorder, Rett's Disorder, Childhood Disintegrative Disorder and Pervasive Developmental Disorder Not Otherwise Specified.

viii) Attention-Deficit/Hyperactivity Disorder (including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9)); Hyperkinetic Disorder; Disruptive Behaviour Disorders such as Conduct Disorder (including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

ix) Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9).

It will be appreciated that references herein to “treatment” extend to prophylaxis, prevention of recurrence and suppression or amelioration of symptoms (whether mild, moderate or severe) as well as the treatment of established conditions. The compound of the invention may be administered as the raw chemical but the active ingredient is suitably presented as a pharmaceutical formulation.

The compounds of the invention may be used in combination with the following agents to treat or prevent male sexual dysfunction: i) phosphodiesterase V inhibitors, for example vardenafil and sildenafil; ii) dopamine agonists/dopamine transport inhibitors for example apomorphine and buproprion; iii) alpha adrenoceptor antagonists for example phentolamine; iv) prostaglandin agonists for example alprostadil; v) testosterone agonists such as testosterone; vi) serotonin reuptake inhibitors for example citalopram, escitalopram, fluoxetine, paroxetine, dapoxetine, sertraline femoxetine, fluvoxamine, indalpine and zimeldine; v) noradrenaline transport inhibitors for example reboxetine.

The compounds of the invention may be used in combination with the same agents specified for male sexual dysfunction to treat or prevent female sexual dysfunction, and in addition an estrogen agonist such as estradiol.

The compounds of the invention may be used in combination with the following agents to treat or prevent psychotic disorders: i) antipsychotics; ii) drugs for extrapyramidal side effects, for example anticholinergics (such as benztropine, biperiden, procyclidine and trihexyphenidyl), antihistamines (such as diphenhydramine) and dopaminergics (such as amantadine); iii) antidepressants; iv) anxiolytics; and v) cognitive enhancers for example cholinesterase inhibitors (such as tacrine, donepezil, rivastigmine and galantamine).

The compounds of the invention may be used in combination with antidepressants to treat or prevent depression and mood disorders.

The compounds of the invention may be used in combination with the following agents to treat or prevent bipolar disease: i) mood stabilisers; ii) antipsychotics; and iii) antidepressants.

The compounds of the invention may be used in combination with the following agents to treat or prevent anxiety disorders: i) anxiolytics; and ii) antidepressants.

Antipsychotic drugs include Typical Antipsychotics (for example chlorpromazine, thioridazine, mesoridazine, fluphenazine, perphenazine, prochlorperazine, trifluoperazine, thiothixine, haloperidol, molindone and loxapine); and Atypical Antipsychotics (for example clozapine, olanzapine, risperidone, quetiapine, aripirazole, ziprasidone and amisulpride).

Antidepressant drugs include serotonin reuptake inhibitors (such as citalopram, escitalopram, fluoxetine, paroxetine, dapoxetine, sertraline femoxetine, fluvoxamine, indalpine and zimeldine); dual serotonin/noradrenaline reuptake inhibitors (such as venlafaxine, duloxetine and milnacipran); Noradrenaline reuptake inhibitors (such as reboxetine and venlafaxine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Mood stabiliser drugs include lithium, sodium valproate/valproic acid/divalproex, carbamazepine, lamotrigine, gabapentin, topiramate and tiagabine.

Anxiolytics include benzodiazepines such as alprazolam and lorazepam.

In addition the compounds of the invention may be administered in combination with 5-HT₃ antagonists (such as ondansetron, granisetron and metoclopramide); serotonin agonists (such as sumatriptan, rauwolscine, yohimbine and metoclopramide); and NK-1 antagonists.

It will be appreciated that the compounds of the combination or composition may be administered simultaneously (either in the same or different pharmaceutical formulations), separately or sequentially.

It will be appreciated that references herein to “treatment” extend to prophylaxis, prevention of recurrence and suppression or amelioration of symptoms (whether mild, moderate or severe) as well as the treatment of established conditions.

The compounds of the invention will normally, but not necessarily, be formulated into pharmaceutical compositions prior to administration to a patient. Accordingly, in another aspect the invention is directed to pharmaceutical compositions comprising a compound of the invention and one or more pharmaceutically-acceptable excipient.

The pharmaceutical compositions of the invention may be prepared and packaged in bulk form wherein a safe and effective amount of a compound of the invention can be extracted and then given to the patient such as with powders or syrups.

Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from 0.01 mg to 50 mg.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as dry powders, aerosols, suspensions, and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweeteners, flavouring agents, flavour masking agents, colouring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

All publications, including, but not limited to, patents and patent applications cited in this specification, are herein incorporated by reference as if each individual publication were specifically and individually indicated to be incorporated by reference herein as though fully set forth.

It will be appreciated that the invention includes the following further aspects. The embodiments described for the first aspect extend these further aspects. The diseases and conditions described above extend, where appropriate, to these further aspects.
i) A compound of the invention for use in treating or preventing sexual dysfunction, such as premature ejaculation.
ii) A compound of the invention for use in enhancing cognition including the treatment of cognition impairment associated with disease.
iii) A method of treatment or prevention of sexual dysfunction (such as premature ejaculation) in a mammal comprising administering an effective amount of a compound of the invention.
iv) A method for enhancing cognition including the treatment of cognition impairment associated with disease in a mammal comprising administering an effective amount of a compound of the invention.

### Supporting Compounds

A number of the compounds of the invention have been prepared and tested in biological assays. These compounds, their preparation and the assays are described below.

In the procedures that follow, after each starting material, reference to an intermediate is typically provided. This is provided merely for assistance to the skilled chemist. The starting material may not necessarily have been prepared from the batch referred to.

Compounds of the invention and intermediates are named using ACD/Name PRO 6.02 chemical naming software (Advanced Chemistry Development Inc., Toronto, Ontario, M5H2L3, Canada).

Proton Magnetic Resonance (NMR) spectra were recorded either on Varian instruments at 300, 400 or 500 MHz, or on a Bruker instrument at 300 or 400 MHz. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one is reported.

Mass spectra (MS) were determined using either
a) a 4 II triple quadrupole Mass Spectrometer (Micromass UK) or on a Agilent MSD 1100 Mass Spectrometer, operating in ES (+) and ES (−) ionization mode or on a Agilent LC/MSD 1100 Mass Spectrometer, operating in ES (+) and ES (−) ionization mode coupled with HPLC instrument Agilent 1100 Series [LC/MS-ES (+): analysis performed on a Supelcosil ABZ +Plus (33×4.6 mm, 3 μm) (mobile phase: 100% [water+0.1% HCO₂H] for 1 min, then from 100% [water+0.1% HCO₂H] to 5% [water+0.1% HCO₂H] and 95% [CH₃CN] in 5 min, finally under these conditions for 2 min; T=40° C.; flux=1 mL/min; LC/MS-ES (−): analysis performed on a Supelcosil ABZ +Plus (33×4.6 mm, 3 μm) (mobile phase: 100% [water+0.05% NH₃] for 1 min, then from 100% [water+0.05% NH₃ to 5% [water+0.05% NH₃] and 95% [CH₃CN] in 5 min, finally under these conditions for 2 min; T=40° C.; flux=1 mL/min]. In the mass spectra only one peak in the molecular ion cluster is reported; or
b) Total ion current (TIC) and DAD UV chromatographic traces together with MS and UV spectra associated with the peaks were taken also on a UPLC/MS Acquity™ system equipped with 2996 PDA detector and coupled to a Waters Micromass ZQ™ mass spectrometer operating in positive or negative electrospray ionisation mode. [LC/MS-ES (+/−): analyses were performed using an Acquity™ HPLC BEH C18 column (50×21 mm, 1.7 μm particle size), column temperature 40° C. (mobile phase: A-water+0.1% HCOOH/B-MeCN+0.075% HCOOH, Flow rate: 1.0 mL/min, Gradient: t=0 min 3% B, t=0.05 min 6% B, t=0.57 min 70% B, t=1.4 min 99% B, t=1.45 min 3% B)].

The optical rotation may be measured on a JASCO DIP-360 digital polarimeter (λ=589 nm, T=20° C., c=1 in MeOH).

Silica solid phase extraction was carried out using either SPE-Si columns supplied by Varian or Isolute Si II supplied by International Supplied Technology.

SPE-SCX cartridges are ion exchange solid phase extraction columns by supplied by Varian. The eluent used with SPE-SCX cartridges is methanol followed by 2N ammonia solution in methanol.

The following table lists the used abbreviations:

| | |
|---|---|
| +/−BINAP | rac-2,2-Bis(diphenylphosphino)-1,1-binaphthyl |
| DCM | dichloromethane |
| DMF | dimethylformamide |
| THF | tetrahydrofuran |

### Compound 1: 6-methyl-5-{2-[4-(2-methyl-5-quinolinyl)-1-piperazinyl]ethyl}-3,4-dihydro-2(1H)-quinolinone dihydrochloride

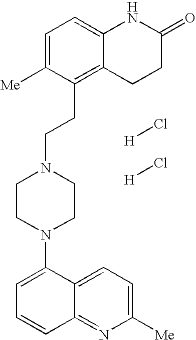

A mixture of (6-methyl-2-oxo-1,2,3,4-tetrahydro-5-quinolinyl)acetaldehyde (Intermediate 1) (70 mg, 0.345 mmol) and 2-methyl-5-(1-piperazinyl)quinoline (WO2004/046124, Description 3) (117 mg, 0.517 mmol) in dry 1,2-dichloroethane (4 ml) was stirred at room temperature for 30 minutes. Sodium triacetoxyborohydride (110 mg, 0.517 mmol) was then added and the resulting reaction mixture was stirred for 4.5 hours. The reaction mixture was quenched with a saturated aqueous solution of NaHCO₃ and extracted with DCM. The combined organics were dried (Na₂SO₄) and concentrated in vacuo. The crude product was purified by SPE cartridge (silica gel, 10 g) eluting with a gradient of methanol in DCM (from 2 to 3%) to afford the free base of the title compound (78 mg, 55%); MS (ES) m/z: 415.2 [MH⁺]; C₂₆H₃₀N₄O requires 414.5. The free base of the title compound (27 mg, 0.07 mmol) was dissolved in methanol/DCM (1/1, 2 ml) and treated with HCl (0.115 ml of a 1.25 M solution in methanol, 0.143 mmol) at 0° C. The resulting mixture was stirred at room temperature for 2 hours. Evaporation of the volatiles and trituration with diethyl ether gave the title compound (27 mg) as a yellow solid; ¹H NMR (500 MHz, DMSO-d₆) δ: 10.95 (br. s., 1H), 10.00 (s, 1H), 8.58 (br. s., 1H), 7.70-7.86 (m, 2H), 7.48-7.68 (m, 1H), 7.22-7.41 (m, 1H), 6.99 (d, 1H), 6.70 (d, 1H), 3.77 (d, 2H), 3.45-3.55 (m, 4H), 3.23-3.34 (m, 2H), 3.15-3.23 (m, 2H), 3.07-3.16 (m, 2H), 2.96 (t, 2H), 2.75 (s, 3H), 2.44 (t, 2H), 2.31 (s, 3H).

The following compounds of formula (Ia) were prepared according to the general reductive amination procedure described for the preparation of Compound 1 starting from the appropriate arylpiperazine or arylpiperidine and (6-methyl-2-oxo-1,2,3,4-tetrahydro-5-quinolinyl)acetaldehyde (Intermediate 1).

| | |
|---|---|
| | (Ia) |
| 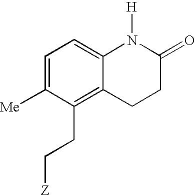 | |

| Cmp | Z | Analytical data ([MH]⁺ Free Base, ¹H NMR) |
|---|---|---|
| 2 | 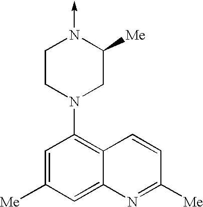 | MS (ES) m/z: 447.2 [MH⁺]; C₂₇H₃₁FN₄O requires 446.5. ¹H NMR (500 MHz, DMSO-d₆) δ: 11.65 (br. s., 1 H), 9.90-10.15 (m, 1 H), 8.82 (br. s., 1 H), 7.70 (br. s., 2 H), 7.35 (br. s., 1 H), 6.89-7.07 (m, 1 H), 6.56-6.81 (m, 1 H), 3.07-4.08 (m, 11 H), 2.92-3.02 (m, 2 H), 2.85 (s, 3 H), 2.41-2.48 (m, 2 H), 2.31 (s, 3 H), 1.36-1.63 (m, 3 H). |
| 3 | 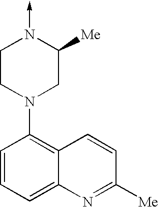 | ¹H NMR (500 MHz, DMSO-d₆) δ: 11.66 (br. s., 1 H), 9.88-10.14 (m, 1 H), 9.02 (br. s., 1 H), 8.04 (br. s., 2 H), 7.85 (br. s., 1 H), 7.46 (br. s., 1 H), 6.87-7.12 (m, 1 H), 6.57-6.80 (m, 1 H), 3.08- 4.10 (m, 11 H), 2.94-3.03 (m, 2 H), 2.93 (s, 3 H), 2.42-2.48 (m, 2 H), 2.32 (s, 3 H), 1.37- 1.64 (m, 3 H). |
| 4 | 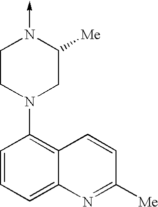 | MS (ES) m/z: 429.2 [MH⁺]; C₂₇H₃₂N₄O requires 428.5. ¹H NMR (500 MHz, DMSO-d₆) δ: 11.83 (br. s., 1 H), 9.91-10.14 (m, 1 H), 9.01 (br. s., 1 H), 8.04 (br. s., 2 H), 7.85 (br. s., 1 H), 7.46 (br. s., 1 H), 6.92-7.09 (m, 1 H), 6.65 -6.77 (m, 1 H), 3.05 - 4.10 (m, 11 H), 2.94-3.01 (m, 2 H), 2.94 (s, 3 H), 2.41-2.49 (m, 2 H), 2.31 (s, 3 H), 1.39- 1.64 (m, 3 H). |
| 5 | 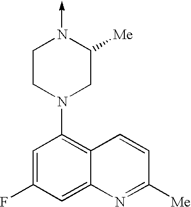 | MS (ES) m/z: 447.2 [MH⁺]; C₂₇H₃₁FN₄O requires 446.5. ¹H NMR (500 MHz, DMSO-d₆) δ: 11.49 (br. s., 1 H), 9.89-10.13 (m, 1 H), 8.77 (br. s., 1 H), 7.65 (br. s., 2 H), 7.32 (br. s., 1 H), 6.88-7.08 (m, 1 H), 6.62-6.76 (m, 1 H), 3.06-4.07 (m, 11 H), 2.91-3.02 (m, 2 H), 2.82 (s, 3 H), 2.41-2.48 (m, 2 H), 2.30 (s, 3 H), 1.35 -1.63 (m, 3 H). |
| 6 | 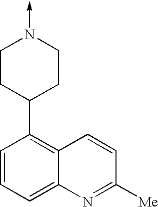 | MS (ES) m/z: 414.2 [MH⁺]; C₂₇H₃₁N₃O requires 413.5. ¹H NMR (500 MHz, DMSO-d₆) δ: 10.91 (br. s., 1 H), 10.00 (s, 1 H), 8.68 (br. s., 1 H), 7.85-7.99 (m, 1 H), 7.74-7.85 (m, 1 H), 7.53 -7.67 (m, 1 H), 7.44-7.53 (m, 1 H), 6.98 (d, 1 H), 6.70 (d, 1 H), 3.79 (d, 2 H), 3.65-3.73 (m, 1 H), 3.23- 3.32 (m, 2 H), 3.04-3.17 (m, 4 H), 2.95 (t, 2 H), 2.73 (s, 3 H), 2.43 (t, 2 H), 2.30 (s, 3 H), 2.15- 2.27 (m, 2 H), 2.04-2.14 (m, 2 H). |

### Compound 7: 1,6-dimethyl-5-{2-[4-(2-methyl-5-quinolinyl)-1-piperazinyl]ethyl}-3,4-dihydro-2(1H)-quinolinone dihydrochloride

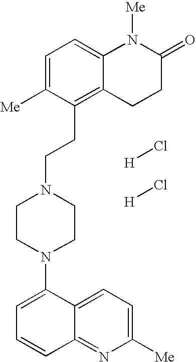

To a solution of 6-methyl-5-{2-[4-(2-methyl-5-quinolinyl)-1-piperazinyl]ethyl}-3,4-dihydro-2(1H)-quinolinone (free base of Compound 1) (50 mg, 0.121 mmol) in dry DMF (3 ml) at 0° C. were added sodium hydride (7 mg of a 60% dispersion in mineral oil, 0.169 mmol) and iodomethane (0.09 ml, 0.145 mmol). After 2 hours the reaction was quenched with water and extracted with DCM. The combined organics were dried (Na₂SO₄) and concentrated in vacuo. The crude product was purified by SPE cartridge (silica gel, 10 g) eluting with a gradient of methanol in DCM (from 2 to 3%) to afford the free base of the title compound (40 mg, 77%); MS (ES) m/z: 429.1 [MH⁺]; C₂₇H₃₂N₄O requires 428.6. The free base (40 mg, 0.09 mmol) was dissolved in methanol (3 ml) and treated with HCl (0.164 ml of a 1.25 M solution in methanol, 0.206 mmol) at 0° C. The resulting mixture was stirred at room temperature for 2 hours. Evaporation of the volatiles and trituration with diethyl ether gave the title compound (40 mg) as a yellow solid; ¹H NMR (500 MHz, DMSO-d₆) δ: 10.77 (br. s., 1H), 8.60 (br. s., 1H), 7.74 (br. s., 2H), 7.54 (br. s., 1H), 7.30 (br. s., 1H), 7.13 (d, 1H), 6.96 (d, 1H), 3.64-3.90 (m, 2H), 3.44-3.61 (m, 4H), 3.22-3.25 (m, 3H), 3.16-3.18 (m, 1H), 3.12-3.40 (m, 6H), 2.84-3.01 (m, 2H), 2.71 (br. s., 3H), 2.47-2.57 (m, 2H), 2.32-2.40 (m, 3H).

The following compounds of formula (Ib) were prepared according to the general reductive amination procedure described for the preparation of Compound 1 starting from the appropriate arylpiperazine and (1,6-dimethyl-2-oxo-1,2,3,4-tetrahydro-5-quinolinyl)acetaldehyde (Intermediate 3).

| | |
|---|---|
| | (Ib) |
| 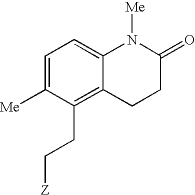 | |

| Cmp | Z | Analytical data ([MH]⁺ Free Base, ¹H NMR) |
|---|---|---|
| 8 | 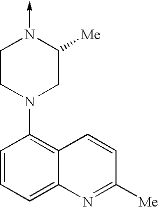 | MS; (ES) m/z: 443.2 [MH⁺]. C₂₈H₃₄N₄O requires 442.6. ¹H NMR (500 MHz, DMSO-d₆) δ: 11.32-11.72 (m, 1 H), 9.01 (br. s., 1 H), 7.75- 8.07 (m, 3 H), 7.45 (br. s., 1 H), 7.05-7.21 (m, 1 H), 6.88-7.02 (m, 1 H), 3.09- 4.09 (m, 14 H), 2.92-3.01 (m, 2 H), 2.85- 2.94 (m, 3 H), 2.50-2.59 (m, 2 H), 2.30-2.42 (m, 3 H), 1.33-1.69 (m, 3 H). |
| 9 | 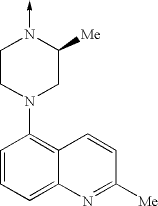 | MS; (ES) m/z: 443.2 [MH⁺]. C₂₈H₃₄N₄O requires 442.6. ¹H NMR (400 MHz, DMSO-d₆) δ: 11.37 (br. s., 1 H), 8.89 (br. s., 1 H), 7.93 (br. s., 2 H), 7.78 (br. s., 1 H), 7.43 (br. s., 1 H), 7.13-7.19 (m, 1 H), 6.95- 7.00 (m, 1 H), 3.17-3.27 (m, 3 H), 3.04-4.10 (m, 11 H), 2.94-3.05 (m, 2 H), 2.83-2.93 (m, 3 H), 2.53-2.60 (m, 2 H), 2.37-2.42 (m, 3 H), 1.42- 1.63 (m, 3 H). |
| 10 | 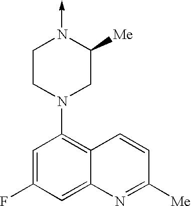 | ¹H NMR (400 MHz, DMSO-d₆) δ: 10.80-11.17 (m, 1 H), 8.57 (br. s., 1 H), 7.39-7.61 (m, 2 H), 7.20- 7.32 (m, 1 H), 7.10-7.21 (m, 1 H), 6.90-7.01 (m, 1 H), 3.10-4.08 (m, 14 H), 2.90-3.03 (m, 2 H), 2.70-2.78 (m, 3 H), 2.54-2.63 (m, 2 H), 2.35- 2.42 (m, 3 H), 1.39-1.63 (m, 3 H). |
| 11 | 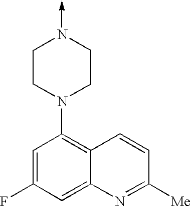 | MS; (ES) m/z: 447.2 [MH⁺]. C₂₇H₃₁FN₄O requires 446.57. ¹H NMR (400 MHz, DMSO-d₆) δ: 10.69 (br. s., 1 H), 8.50 (d, 1 H) 7.52 (d, 1 H), 7.46 (d, 1 H), 7.25 (d, 1 H), 7.15 (d, 1 H), 6.97 (d, 1 H), 3.23-3.27 (m, 3 H), 3.09-3.86 (m, 12 H), 2.93-3.00 (m, 2 H), 2.70- 2.75 (m, 3 H), 2.55-2.56 (m, 2 H), 2.36-2.40 (m, 3H). |
| 12 | 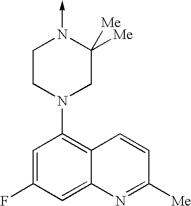 | ¹H NMR (500 MHz, DMSO-d₆) δ: 1.44-1.61 (m, 6 H), 2.30-2.39 (m, 3 H), 2.47-2.59 (m, 2 H), 2.73- 3.00 (m, 7 H), 3.16-3.96 (m, 11 H), 6.95 (d, 1 H), 7.13 (d, 1 H), 7.34 (br. s., 1 H), 7.66 (br. s., 1 H), 7.83 (br. s., 2 H), 8.75 (br. s., 1 H), 10.56 (br. s., 1 H). |

### Compound 13: 1,6-dimethyl-5-{2-[4-(2-methyl-5-quinolinyl)-1-piperidinyl]ethyl}-3,4-dihydro-2(1H)-quinolinone dihydrochloride

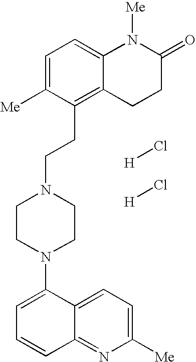

The title compound was prepared in a similar fashion to the preparation of Compound 7 starting from free base of Compound 6. The free base of the title compound gave MS (ES) m/z: 428.3 [MH⁺]; C₂₈H₃₃N₃O requires 427.6; ¹H NMR (of the salt) (300 MHz, DMSO-d₆) δ: 11.16 (br. s., 1H), 9.08 (br. s., 1H), 8.3-7.5 (m, 4H), 7.11 (d, 1H), 6.92 (d, 1H), 4.0-2.8 (m, 17H), 2.40-2.0 (t, 9H).

The following compounds of formula (Ic) were prepared according to the general reductive amination procedure described for the preparation of Compound 1 starting from the appropriate arylpiperazine or arylpiperidine and (6-methyl-2-oxo-1,2-dihydro-5-quinolinyl)acetaldehyde (Intermediate 14).

| | |
|---|---|
| | (Ic) |
| 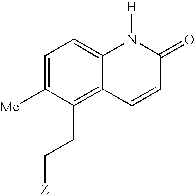 | |

| Cmp | Z | Analytical data ([MH]⁺ Free Base, ¹H NMR) |
|---|---|---|
| 14 | 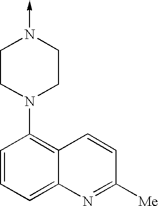 | MS (ES) m/z: 413.1 [MH⁺]; C₂₈H₂₈N₄O requires 412.53. ¹H NMR (500 MHz, DMSO-d₆) δ: 11.76 (s, 1 H), 11.06 (br. s., 1 H), 8.45 (br. s., 1 H), 8.26 (d, 1 H), 7.70 (m, 2 H), 7.49 (br. s., 1 H), 7.37 (d, 1 H), 7.26 (br. s., 1 H), 718 (d, 1 H), 6.55 (d, 1 H), 3.76-3.88 (m, 2 H), 3.20-3.61 (m, 10 H), 2.68 (br. s., 3 H), 2.42 (5, 3 H). |
| 15 | 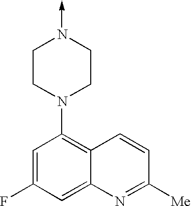 | MS (ES) m/z: 431.2 [MH⁺]; C₂₆H₂₇FN₄O requires 430.52. ¹H NMR (500 MHz, DMSO-d₆) δ: 11.77 (s,1 H), 11.15 (br. s., 1 H), 8.60 (br. s., 1 H), 8.27 (d, 1 H), 7.62 (d, 1 H), 7.52 (d, 1 H), 7.40 (d, 1 H), 7.32 (d, 1 H), 7.21 (d, 1 H), 6.58 (d, 1 H), 3.84 (d, 2 H), 3.20- 3.70 (m, 10 H), 2.78 (br. s., 3 H), 2.44 (s, 3 H). |
| 16 | 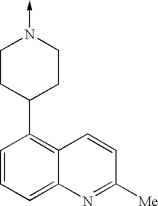 | MS (ES) m/z: 412.1 [MH⁺]; C₂₇H₂₉N₃O requires 411.55. ¹H NMR (500 MHz, DMSO-d₆) δ: 11.74-11.78 (m, 1 H), 10.88 (br. s., 1 H), 8.76 (br. s., 1 H), 8.25 (d, 1 H), 7.44-8.05 (m, 4 H), 7.38 (d, 1 H), 7.18 (d, 1 H), 6.55 (d, 1 H), 3.79-3.89 (m, 2 H), 3.69-3.78 (m, 1 H), 3.23-3.50 (m, 4 H), 3.11-3.21 (m, 2 H), 2.70- 2.83 (m, 3 H), 2.38-2.44 (m, 3 H), 2.07-2.30 (m, 4 H). |

The following compounds of formula (Id) were prepared according to the general reductive amination procedure described for the preparation of Compound 1 starting from the appropriate arylpiperazine or arylpiperidine and (1,6-dimethyl-2-oxo-1,2-dihydro-5-quinolinyl)acetaldehyde (Intermediate 12).

| | |
|---|---|
| | (Id) |
| 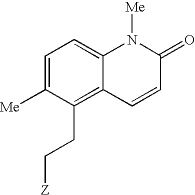 | |

| Cmp | Z | Analytical data ([MH]⁺ Free Base, ¹H NMR) |
|---|---|---|
| 17 | 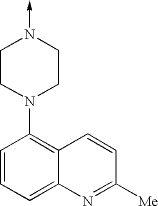 | MS (ES) m/z: 427.1 [MH⁺]; C₂₇H₃₀N₄O requires 426.6. ¹H NMR (500 MHz, DMSO-d₆) δ: 10.93 (br. s., 1 H), 8.34-8.65 (m, 1 H), 8.26 (d, 1 H), 7.66-7.78 (m, 2 H), 7.52 (d, 2 H), 7.42 (d, 1 H), 7.21-7.34 (m, 1 H), 6.68 (d, 1 H), 3.76-3.88 (m, 2 H), 3.62 (s, 3 H), 3.31-3.61 (m, 8 H), 3.26-3.38 (m, 2 H), 2.64-2.75 (m, 3 H), 2.46 (s, 3 H). |
| 18 | 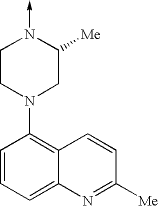 | MS (ES) m/z: 441.1 [MH⁺]; C₂₈H₃₂N₄O requires 440.6. ¹H NMR (500 MHz, DMSO-d₆) δ: 11.80 (br. s., 1 H), 8.84-9.11 (m, 1 H), 8.31 (d, 1 H), 7.91-8.09 (m, 2 H), 7.85 (d, 1 H), 7.45-7.60 (m, 2 H), 7.41 (d, 1 H), 6.67 (d, 1 H), 3.91-4.10 (m, 2 H), 3.67- 3.89 (m, 2 H), 3.61 (s, 3 H), 3.04-3.72 (m, 7 H), 2.91 (s, 3 H), 2.49 (s, 3 H), 1.46 (d, 3 H). |
| 19 | 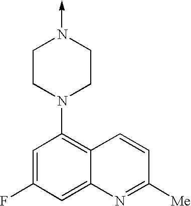 | MS (ES) m/z: 445.1 [MH⁺]; C₂₇H₂₉N₄O requires 444.6. ¹H NMR (500 MHz, DMSO-d₆) δ: 11.48 (br. s., 1 H), 8.53-8.74 (m, 1 H), 8.31 (d, 1 H), 7.62 (d, 1 H), 7.56 (d, 1 H), 7.51 (d, 1 H), 7.42 (d, 1 H), 7.31 (d, 1 H), 6.67 (d, 1 H), 3.82 (d, 2 H), 3.61 (s, 3 H), 3.17-3.66 (m, 10 H), 2.77 (5, 3 H), 2.46 (5, 3 H). |
| 20 | 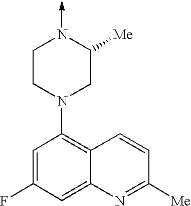 | MS (ES) m/z: 459.2 [MH⁺]; C₂₈H₃₁FN₄O requires 458.6. ¹H NMR (500 MHz, DMSO-d₆) δ: 11.69 (br. s., 1 H), 8.79 (br. s., 1 H), 8.30 (d, 1 H), 7.55-7.78 (m, 2 H), 7.52 (d, 1 H), 7.43 (d, 1 H), 7.28-7.39 (m, 1 H), 6.67 (d, 1 H), 3.62 (br. s., 3 H), 3.04-4.38 (m, 11 H), 2.83 (br. s., 3 H), 2.49 (s, 3 H), 1.46 (d, 3 H). |
| 21 | 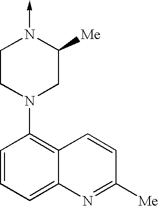 | MS (ES) m/z: 441.3 [MH⁺]; C₂₈H₃₂N₄O requires 440.5. ¹H NMR (500 MHz, DMSO-d₆) δ: 11.75 (br. s., 1 H), 8.97 (br. s., 1 H), 8.21-8.50 (m, 1 H), 7.98 (br. s., 2 H), 7.85 (br. s., 1 H), 7.46-7.56 (m, 2 H), 7.39-7.46 (m, 1 H), 6.60-6.73 (m, 1 H), 3.61 (s, 3 H), 3.04-4.10 (m, 11 H), 2.90 (5, 3 H), 2.47 (s, 3 H), 1.41-1.62 (m, 3 H). |
| 22 | 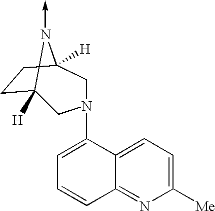 | ¹H NMR (400 MHz, DMSO-d6) δ: 12.05 (br. s., 1 H), 9.01 (bd, 1 H) 8.45 (d, 1H) 8.1 (m, 2H)-7.86 (m, 1 H) 7.55-7.68 (m, 2 H) 7.45 (d, 1 H), 6.75 (d, 1 H) 6.70 (d, 1 H) 4.5 (m, 2 H) 3.44-3.61 (m, 4 H) 3.22-3.25 (m, 3 H) 3.16-3.18 (m, 1 H) 3.12- 3.40 (m, 6 H) 2.84-3.01 (m, 2 H) 2.71 (br. s., 3 H) 2.47-2.57 (m, 2H) 2.32-2.40 (m, 3 H). |
| 23 | 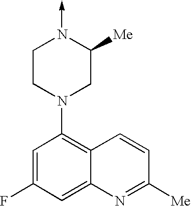 | MS (ES) m/z: 459.2 [MH⁺]; C₂₈H₃₁FN₄O requires 458.5. ¹H NMR (500 MHz, DMSO-d₆) δ: 11.66 (br. s., 1 H), 8.77 (br. s., 1 H), 8.21-8.48 (m, 1 H), 7.57- 7.76 (m, 2 H), 7.51 (d, 1 H), 7.43 (d, 1 H), 7.35 (br. s., 1 H), 6.60-6.75 (m, 1 H), 3.62 (s, 3 H), 3.08-4.13 (m, 11 H), 2.82 (s, 3 H), 2.47 (s, 3 H), 1.38-1.62 (m, 3 H). |
| 24 | 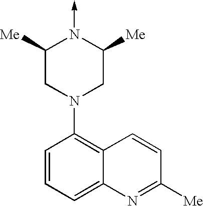 | ¹H NMR (500 MHz, DMSO-d₆) δ: 11.22 (s, 1 H), 8.94-9.01 (m, 1 H), 7.67-8.23 (m, 4 H), 7.20- 7.65 (m, 3 H), 6.56-6.85 (m, 1 H), 4.02-4.28 (m, 2 H), 3.06-3.75 (m, 14 H), 2.77-3.04 (m, 3 H), 1.37-1.70 (m, 6 H). |
| 25 | 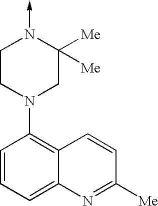 | MS (ES) m/z: 455 [MH⁺]; C29H34N4O requires 454.61 ¹H NMR (500 MHz, DMSO-d₆) δ: 1.50-1.58 (m, 6 H), 2.44-2.48 (m, 3 H), 2.86 (br. s., 3 H), 2.92- 3.05 (m, 2 H), 3.21-4.03 (m, 8 H), 3.60-3.64 (m, 3 H), 6.69 (d, 1 H), 7.43 (br. s., 1 H), 7.43 (d, 1 H), 7.53 (d, 1 H), 7.79 (br. s., 1 H), 7.91 (br. s., 2 H), 8.26 (d, 1 H), 8.90 (br. s., 1 H), 11.16 (br. s., 1 H). |
| 26 | 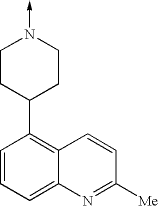 | MS (ES) m/z: 426.2 [MH⁺]; C28H31N3O requires 425.57; ¹H NMR (500 MHz, DMSO-d₆) δ: 10.52 (br. s., 1 H), 8.49-8.75 (m, 1 H), 8.23 (d, 1 H), 7.80-7.95 (m, 1 H), 7.68-7.82 (m, 1 H), 7.49- 7.57 (m, 2 H), 7.44-7.50 (m, 1 H), 7.42 (d, 1 H), 6.67 (d, 1 H), 3.80-3.90 (m, 2 H), 3.64-3.76 (m, 1 H), 3.62 (s, 3 H), 3.40-3.51 (m, 2 H), 3.25- 3.40 (m, 2 H), 3.11-3.24 (m, 2 H), 2.69 (s, 3 H), 2.46 (s, 3 H), 2.05-2.25 (m, 4 H). |

The following compounds of formula (Ie) were prepared according to the general reductive amination procedure described for the preparation of Compound 1 starting from the appropriate arylpiperazine or arylpiperidine and (6-chloro-1-methyl-2-oxo-1,2-dihydro-5-quinolinyl)acetaldehyde (Intermediate 23).

| | |
|---|---|
| | (Id) |
| 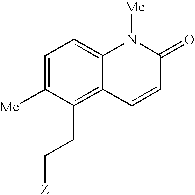 | |

| Cmp | Z | Analytical data ([MH]⁺ Free Base, ¹H NMR) |
|---|---|---|
| 27 | 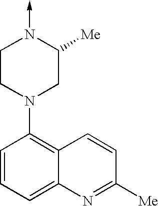 | MS (ES) m/z: 461.2 [MH⁺]; C₂₇H₂₉ClN₄O requires 461.01. ¹H NMR (500 MHz, DMSO-d₆) δ: 11.84 (br. s., 1 H), 8.67-9.05 (m, 1 H), 8.38 (d, 1 H), 7.78- 8.06 (m, 2 H), 7.73 (d, 1 H), 7.62-7.81 (m, 1 H), 7.57 (d, 1 H), 7.28-7.50 (m, 1 H), 6.75 (d, 1 H), 3.64 (s, 3 H), 3.07-4.03 (m, 11 H), 2.86 (s, 3 H), 1.46 (d, 3 H). |
| 28 | 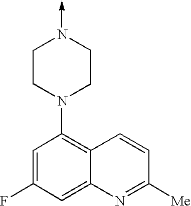 | MS (ES) m/z: 465.1 [MH⁺]; C₂₆H₂₆ClFN₄O requires 464.97. ¹H NMR (500 MHz, DMSO-d₆) δ: 11.88 (br. s., 1 H), 8.53-8.81 (m, 1 H), 8.41 (d, 1 H), 7.73 (d, 1 H), 7.56 (d, 1 H), 7.52-7.67 (m, 2 H), 7.31 (d, 1 H), 6.74 (d, 1 H), 3.83 (d, 2 H), 3.63 (5, 3 H), 3.22-3.74 (m, 10 H), 2.79 (s, 3 H). |
| 29 | 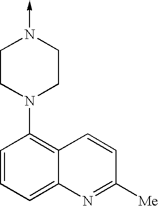 | MS (ES) m/z: 447.2 [MH⁺]; C₂₆H₂₇ClN₄O requires 446.98. ¹H NMR (500 MHz, DMSO-d₆) δ: 11.82 (br. s., 1 H), 8.60-8.98 (m, 1 H), 8.41 (d, 1 H), 7.78- 8.03 (m, 2 H), 7.75 (d, 1 H), 7.65-7.81 (m, 1 H), 7.57 (d, 1 H), 7.29-7.51 (m, 1 H), 6.75 (d, 1 H), 3.81 (d, 2 H), 3.63 (s, 3 H), 3.17-3.71 (m, 10 H), 2.84 (s, 3 H). |
| 30 | 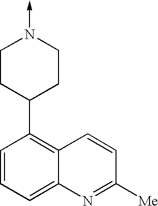 | MS (ES) m/z: 446.2 [MH⁺]; C₂₇H₂₈ClN₃O requires 445.99. ¹H NMR (500 MHz, DMSO-d₆) δ: 11.02 (br. s., 1 H), 8.61-9.22 (m, 1 H), 8.37 (d, 1 H), 7.94- 8.09 (m, 1 H), 7.82-7.97 (m, 1 H), 7.74 (d, 1 H), 7.66-7.81 (m, 1 H), 7.56 (d, 1 H), 7.48-7.67 (m, 1 H), 6.78 (d, 1 H), 3.84 (d, 2 H), 3.71-3.83 (m, 1 H), 3.65 (s, 3 H), 3.57-3.66 (m, 2 H), 3.23- 3.46 (m, 2 H), 3.15-3.31 (m, 2 H), 2.81 (s, 3 H), 2.14-2.29 (m, 2 H), 2.02-2.18 (m, 2 H). |

### Compounds 31 and 32: Separated enantiomers of 3,6-dimethyl-7-{2-[4-(2-methyl-5-quinolinyl)-1-piperazinyl]ethyl}1,1a,3,7b-tetrahydro-2H-cyclopropa[c]quinolin-2-one dihydrochloride

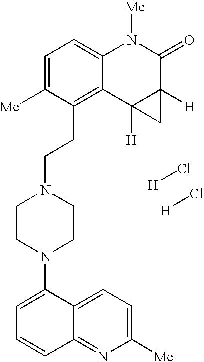

The free bases of the title compounds were prepared as a racemic mixture according to the general reductive amination procedure (Example 1) starting from the racemic mixture 2-methyl-5-(1-piperazinyl)quinoline and (3,6-dimethyl-2-oxo-1a,2,3,7b-tetrahydro-1H-cyclopropa[c]quinolin-7-yl)acetaldehyde (Intermediate 26); MS: (ES) m/z: 441 [MH⁺]. C₂₈H₃₄N₄O requires 440.59. The free bases were separated by semi preparative HPLC chromatography CHIRALCEL OD, 25×2.1 cm; mobile phase (n-Hexane, modifier: 30% Ethanol), flow rate=13 ml/min; UV wavelength: 225 nm; to obtain the free base of Compound 31 (enantiomer 1, 29 mg) and the free base of Compound 32 (enantiomer 2 (28 mg). The enantiomeric excess of both enantiomers were verified by Analytical HPLC conditions: Chiral column: CHIRALCEL OD, 25×0.46 cm; mobile phase (n-Hexane, modifier: 28% Ethanol), flow rate=0.8 ml/min; UV wavelength: DAD (210-340 nm); CD=230 nm (circular dichroism). The free base of Compound 31 (Enantiomer 1): >99.5% a/a by UV, retention time 8.8 min, e.e. >99.5%; MS: (ES) m/z: 441 [MH⁺]. C₂₈H₃₄N₄O requires 440.59. The free base of Compound 32 (Enantiomer 2): 96.8% a/a by UV, retention time 10.6 min; e.e. =93.74%; contains 3.1% of Enantiomer 1; MS: (ES) m/z: 441 [MH⁺]. C₂₈H₃₄N₄O requires 440.59.

The free bases were converted to the hydrochloride salts in the usual way. Compound 31 (Enantiomer 1): ¹H NMR (400 MHz, DMSO-d₆) δ: 11.61 (br. s., 1H), 8.93 (br. s., 1H), 7.91-8.04 (m, 2H), 7.77-7.89 (m, 1H), 7.48 (br. s., 1H), 7.10 (d, 1H), 6.91 (d, 1H), 3.23-4.02 (m, 12H), 3.19-3.23 (m, 3H), 2.89-2.96 (m, 3H), 2.81-2.91 (m, 1H), 2.34-2.40 (m, 3H), 2.14-2.22 (m, 1H), 1.66-1.74 (m, 1H), 0.47-0.54 (m, 1H).

Compound 32 (Enantiomer 2): ¹H NMR (400 MHz, DMSO-d₆) δ: 11.64 (br. s., 1H), 8.95 (br. s., 1H), 7.91-8.10 (m, 2H), 7.77-7.90 (m, 1H), 7.48 (br. s., 1H), 7.10 (d, 1H), 6.92 (d, 1H), 3.24-3.93 (m, 12H), 3.19-3.24 (m, 3H), 2.91-2.95 (m, 3H), 2.81-2.91 (m, 1H), 2.34-2.40 (m, 3H), 2.13-2.21 (m, 1H), 1.63-1.74 (m, 1H), 0.46-0.54 (m, 1H).

### Compounds 33 and 34: Separated diastereoisomers of 3,6-dimethyl-7-{2-[(2S)-2-methyl-4-(2-methyl-5-quinolinyl)-1-piperazinyl]ethyl}1,1a,3,7b-tetrahydro-2H-cyclopropa[c]quinolin-2-one dihydrochloride

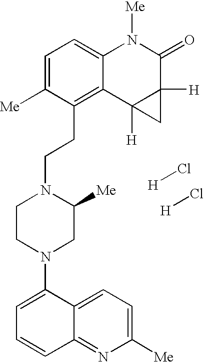

The free bases of the title compounds were prepared as a mixture of diastereoisomers according to the general reductive amination procedure (Example 1) starting from 2-methyl-5-[(3S)-3-methyl-1-piperazinyl]quinoline and (3,6-dimethyl-2-oxo-1a,2,3,7b-tetrahydro-1H-cyclopropa[c]quinolin-7-yl)acetaldehyde (Intermediate 26); MS: (ES) m/z: 455 [MH⁺]. C₂₈H₃₄N₄O requires 454.6. The mixture was separated by semi-preparative HPLC chromatography CHIRALPAK AD-H, 25×2.1 cm; mobile phase (n-Hexane, modifier: 45% Ethanol), flow rate=11 ml/min; UV wavelength: 225 nm; to obtain the free base of Compound 33 (diastereoisomer 1) (38 mg) and the free base of Compound 34 (diastereoisomer 2) (31 mg). The free bases were converted to the hydrochloride salts in the usual way. The enantiomeric purity of the diastereoisomers were verified by Analytical HPLC conditions: Chiral column: CHIRALPAK AD-H, 25×0.46 cm; mobile phase (n-Hexane, modifier: 45% Ethanol), flow rate=0.8 ml/min; UV wavelength: DAD (210-340 nm); CD=230 nm (circular dichroism).

Compound 33 (Diastereoisomer 1): >99.5% a/a by UV, retention time 10.1 min (no trace of Compound 34 (diastereoisomer 2) was detected); ¹H NMR (500 MHz, DMSO-d₆) δ: 11.33 (br. s., 1H), 8.89 (br. s., 1H), 7.97 (br. s., 2H), 7.78 (br. s., 1H), 7.42 (br. s., 1H), 7.00-7.19 (m, 1H), 6.89-6.94 (m, 1H), 3.19 (br. s., 3H), 3.08-4.13 (m, 11H), 2.89 (br. s., 3H), 2.75-2.88 (m, 1H), 2.28-2.40 (m, 3H), 2.09-2.24 (m, 1H), 1.61-1.74 (m, 1H), 1.39-1.59 (m, 3H), 0.44-0.59 (m, 1H).

Compound 34 (Diastereoisomer 2): >99.5% a/a by UV, retention time 14.6 min (no trace of Compound 33 (diastereoisomer 1) was detected); ¹H NMR (500 MHz, DMSO-d₆) δ: 11.13-11.79 (m, 1H), 8.92 (br. s., 1H), 7.94 (br. s., 2H), 7.81 (br. s., 1H), 7.44 (br. s., 1H), 7.06-7.14 (m, 1H), 6.88-6.95 (m, 1H), 3.21 (s, 3H), 3.11-4.14 (m, 11H), 2.89 (br. s., 3H), 2.78-2.87 (m, 1H), 2.31-2.41 (m, 3H), 2.13-2.22 (m, 1H), 1.61-1.71 (m, 1H), 1.40-1.63 (m, 3H), 0.45-0.56 (m, 1H).

### Compounds 35 and 36: Separated Diastereomers of 3,6-dimethyl-7-{2-[(2R)-2-methyl-4-(2-methyl-5-quinolinyl)-1-piperazinyl]ethyl}-1,1a,3,7b-tetrahydro-2H-cyclopropa[c]quinolin-2-one Dihydrochloride

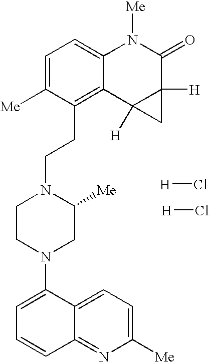

The free bases of the title compounds were prepared as a mixture of diastereoisomers according to the general reductive amination procedure (Example 1) starting from 2-methyl-5-[(3R)-3-methyl-1-piperazinyl]quinoline and (3,6-dimethyl-2-oxo-1a, 2,3,7b-tetrahydro-1H-cyclopropa[c]quinolin-7-yl)acetaldehyde (Intermediate 26); MS: (ES) m/z: 455 [MH⁺]. C₂₈H₃₄N₄O requires 454.6. The mixture was separated by semi-preparative HPLC chromatography CHIRALCEL OD, 25×2.1 cm; mobile phase (n-Hexane, modifier: 30% Isopropanol), flow rate=15 ml/min; UV wavelength: 220 nm; to obtain the free base of Compound 35 (diastereoisomer 1) (40 mg) and the free base of Compound 36 (diastereoisomer 2) (37 mg). The free bases were converted to the hydrochloride salts in the usual way. The enantiomeric purity of the diastereoisomers were verified by Analytical HPLC conditions: Chiral column: CHIRALCEL OD, 25×0.46 cm; mobile phase (n-Hexane, modifier: 25% isopropanol), flow rate=0.9 ml/min; UV wavelength: DAD (210-340 nm); CD=225 nm (circular dichroism).

Compound 35 (Diastereoisomer 1): 99.8% a/a by UV, retention time 11.8 min (no Compound 36 (diastereoisomer 2) detected); ¹H NMR (500 MHz, DMSO-d₆) δ: 11.62-11.88 (m, 1H), 8.99 (br. s., 1H), 8.00 (br. s., 2H), 7.84 (br. s., 1H), 7.47 (br. s., 1H), 7.05-7.16 (m, 1H), 6.86-6.97 (m, 1H), 3.20 (br. s., 3H), 3.08-4.12 (m, 11H), 2.93 (br. s., 3H), 2.79-2.92 (m, 1H), 2.32-2.41 (m, 3H), 2.13-2.24 (m, 1H), 1.62-1.75 (m, 1H), 1.42-1.60 (m, 3H), 0.46-0.59 (m, 1H).

Compound 36 (Diastereoisomer 2): (99.8% a/a by UV, retention time 18.3 min (no Compound 35 (diastereoisomer 1) detected); ¹H NMR (500 MHz, DMSO-d₆) δ: 11.53-12.13 (m, 1H), 9.05 (br. s., 1H), 7.94-8.11 (m, 2H), 7.87 (br. s., 1H), 7.41-7.58 (m, 1H), 7.05-7.16 (m, 1H), 6.84-6.98 (m, 1H), 3.16-3.24 (m, 3H), 3.14-4.13 (m, 11H), 2.95 (br. s., 3H), 2.80-2.92 (m, 1H), 2.31-2.43 (m, 3H), 2.10-2.24 (m, 1H), 1.60-1.72 (m, 1H), 1.42-1.63 (m, 3H), 0.44-0.55 (m, 1H).

### Intermediate 1: (6-methyl-2-oxo-1,2,3,4-tetrahydro-5-quinolinyl)acetaldehyde

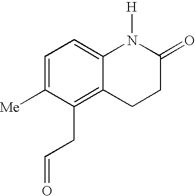

Osmium tetroxide (1.4 ml of a 4% by wt. solution in water, 0.125 eq) was added to a stirred solution of 6-methyl-5-(2-propen-1-yl)-3,4-dihydro-2(1H)-quinolinone (for preparation see WO2006024517, Description 123) (350 mg, 1.74 mmol) in THF/water (2:1; 30 ml). After 10 minutes, sodium periodate (930 mg, 4.35 mmol) was added and the reaction mixture was stirred for 2 hours. After evaporation of THF the residue was partitioned between water and ethyl acetate. The organic layers were combined, dried (Na₂SO₄) and concentrated in vacuo. The crude product was purified by SPE-Si cartridge, eluting with ethyl acetate/cyclohexane (7/3) to afford the title compound (219 mg, 62%); ¹H-NMR (300 MHz, CDCl₃) δ: 9.7 (t, 1H), 7.04 (d, 1H), 6.60 (d, 1H), 3.80 (d, 2H), 2.88 (t, 2H), 2.60 (t, 2H), 2.27 (s, 3H).

### Intermediate 2: 1,6-dimethyl-5-(2-propen-1-yl)-3,4-dihydro-2(1H)-quinolinone

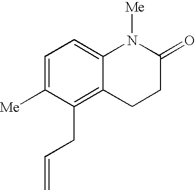

To a solution of 6-methyl-5-(2-propen-1-yl)-3,4-dihydro-2(1H)-quinolinone (for preparation see WO2006024517, Description 123) (400 mg, 1.99 mmol) in dry DMF (2 ml) at 0° C., were added sodium hydride (88 mg of a 60% dispersion in mineral oil, 2.19 mmol) and iodomethane (0.136 ml, 2.19 mmol). After 3 hours the reaction mixture was quenched with saturated aqueous NH₄Cl and extracted with ethyl acetate. The combined organic extracts were washed with water and brine, dried (Na₂SO₄) and concentrated in vacuo. The crude product was purified by flash chromatography on silica gel eluting with ethyl acetate/cyclohexane (1/4) to afford the title compound as pale orange oil (326 mg, 76%); MS; (ES) m/z: 216.2 [MH⁺]. C₁₄H₁₇NO requires 215.29.

### Intermediate 3: (1,6-dimethyl-2-oxo-1,2,3,4-tetrahydro-5-quinolinyl)acetaldehyde (D3)

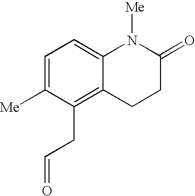

The title compound was prepared in 65% yield in a similar fashion to the preparation of Intermediate 1 starting from 1,6-dimethyl-5-(2-propen-1-yl)-3,4-dihydro-2(1H)-quinolinone (Intermediate 2, 325 mg, 1.51 mmol); ¹H-NMR (400 MHz, CDCl₃) δ; 9.74 (t, 1H), 7.16 (d, 1H), 6.91 (d, 1H), 3.85 (d, 2H), 3.35 (s, 3H), 2.80-2.86 (m, 2H), 2.59-2.65 (m, 2H), 2.31 (s, 3H).

### Intermediate 4: 5-[(3R,5S)-3,5-dimethyl-1-piperazinyl]-2-methylquinoline

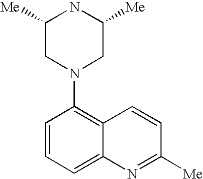

To a solution of 2-methyl-5-quinolinyl trifluoromethanesulfonate (for a preparation see WO2004046124, Description 1; 0.5 g, 1.72 mmol) in dry toluene (20 ml) were added cis-2,6-dimethylpiperazine (0.2 g, 1.8 mmol), caesium carbonate (0.84 g, 2.58 mmol), palladium (II) acetate (0.02 g, 0.086 mmol) and BINAP (0.214 g, 0.344 mmol). The reaction mixture was heated at 90° C. for 18 hours, the solvent evaporated and the crude product purified by silica flash chromatography (DCM/MeOH 95:5) to give the title compound (0.2 g) as a brown oil; ¹H-NMR (400 MHz, CDCl₃) δ: 8.8 (d, 1H), 7.8 (d, 1H), 7.65 (t, 1H), 7.32 (d, 1H), 7.05 (d, 1H), 3.38 (m, 2H), 3.25 (d, 2H), 2.60 (t, 2H), 2.31 (s, 3H), 1.1 (s, 6H).

### Intermediate 5: 5-(3,3-dimethyl-1-piperazinyl)-2-methylquinoline

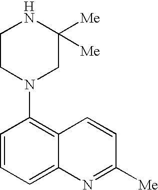

The title compound was prepared in a similar way to Intermediate 4 starting from 2-methyl-5-quinolinyl trifluoromethanesulfonate and 2,2-dimethylpiperazine (commercially available); MS (ES) m/z: 256 [MH⁺].

### Intermediate 6: 5-(3,8-diazabicyclo[3.2.1]oct-3-yl)-2-methylquinoline

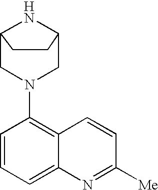

The title compound was prepared by reaction of 2-methyl-5-quinolinyl trifluoromethanesulfonate with 1,1-dimethylethyl 3,8-diazabicyclo[3.2.1]octane-8-carboxylate according to the procedure described for Intermediate 4, followed by removal of the t-butoxycarbonyl group by treatment with trifluoroacetic acid in DCM (1:1) at room temperature for 1 hour. The solvent was removed and the crude product was purified using a SCX cartridge eluting with NH₄OH/MeOH 98/2; MS (ES) m/z: 254 [MH⁺].

### Intermediate 7: 7-fluoro-2-methyl-5-[(3S)-3-methyl-1-piperazinyl]quinoline

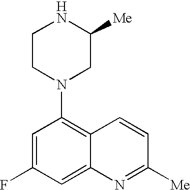

The title compound was prepared in a similar way to the preparation of Intermediate 4 from 7-fluoro-2-methyl-5-quinolinyl trifluoromethanesulfonate (for preparation see WO2004046124, Description 101) and (2S)-2-methylpiperazine; ¹H-NMR (400 MHz, CDCl₃) δ: 8.31 (d, 1H), 7.29-7.42 (m, 1H), 7.22 (d, 1H), 6.76-6.88 (m, 1H), 3.12-3,31 (m, 5H), 2.76-2.91 (m, 1H), 2.66-2.76 (m, 3H), 2.42-2.56 (m, 1H), 1.09-1.21 (m, 3H).

### Intermediate 8: 5-bromo-6-methylquinoline 1-oxide

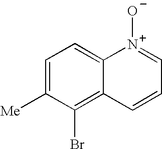

3-Chloroperbenzoic acid (77%, 24.1 g, 139.4 mmol) was added to a solution of 5-bromo-6-methylquinoline (19.9 g, 89.5 mmol, for preparation see Chem. Heterocycl. Compd. (Engl. Trans.) 1998 vol 24, 8, 892) in DCM (331 ml) at 0° C. The reaction mixture was stirred at room temperature for 2 hours and then quenched with saturated aqueous NaHCO₃. The reaction mixture was extracted with DCM and the combined organic extracts were washed again with saturated aqueous NaHCO₃, then dried (Na₂SO₄) and evaporated in vacuo, to give the title compound as a solid (21.30 g, 100%); MS; (ES) m/z: 238, 240 [MH⁺]. C₁₀H₈BrNO requires 238.08; ¹H-NMR (400 MHz, CDCl₃) δ: 8.67 (d, 1H), 8.54 (d, 1H), 8.19 (d, 1H), 7.65 (d, 1H), 7.38 (dd, 1H), 2.66 (s, 3H).

### Intermediate 9: 5-bromo-6-methyl-2(1H)-quinolinone

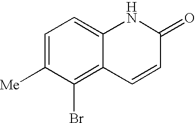

Trifluoroacetic anhydride (48.5 ml, 348.6 mmol) was added to a solution of 5-bromo-6-methylquinoline 1-oxide (Intermediate 8) (16.6 g, 69.7 mmol) in DMF (46 ml) at 0° C. The reaction mixture was stirred at room temperature overnight, then poured into saturated aqueous NaHCO₃ (600 ml). The resulting precipitate was filtered off and triturated with diethyl ether to give the title compound (13.5 g, 81%); MS: (ES) m/z: 238, 240 [MH⁺]. C₁₀H₈BrNO requires 238.08; ¹H-NMR (400 MHz, CDCl₃) δ: 11.92 (br.s., 1H), 8.08 (d, 1H), 7.47 (d, 1H), 7.24 (d, 1H), 6.60 (d, 1H), 2.42 (s, 3H).

### Intermediate 10: 5-bromo-1,6-dimethyl-2(1H)-quinolinone

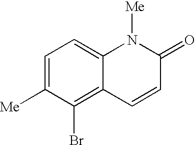

To a solution of 5-bromo-6-methyl-2(1H)-quinolinone (Intermediate 9) (7.0 g, 29.4 mmol) in dry DMF (100 ml) at 0° C. were added sodium hydride (1.65 g of a 60% dispersion in mineral oil, 41.2 mmol) and iodomethane (2.2 ml, 35.3 mmol). The reaction mixture was stirred at room temperature for 1 hour then quenched with water and extracted with DCM. The combined organics were dried (Na₂SO₄) and concentrated in vacuo. The crude product was triturated with diethyl ether to afford the title compound (4.98 g, 67%); MS; (ES) m/z: 252, 254 [MH⁺]. C₁₁H₁₀BrNO requires 252.11.

### Intermediate 11: 1,6-dimethyl-5-(2-propen-1-yl)-2(1-quinolinone

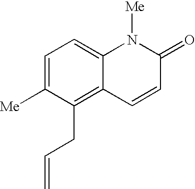

To a solution of 5-bromo-1,6-dimethyl-2(1H)-quinolinone (Intermediate 10) (4.98 g, 19.76 mmol) in dry DMF (100 ml) were added allyltributylstannane (7.85 g, 23.7 mmol), palladium (0) tetrakistriphenylphosphine (2.28 g, 1.98 mmol) and lithium chloride (0.016 g, 0.4 mmol). The reaction mixture was heated at 100° C. for 1.5 hours, then diluted with water and extracted with DCM. The organic layers were combined, dried (Na₂SO₄) and evaporated in vacuo. The crude material was purified by silica flash chromatography ethyl acetate/cyclohexane (7:3) to afford the title compound (3.95 g, 94%); MS (ES) m/z: 214.17 [MH⁺]; C₁₄H₁₅NO requires 213.28.

### Intermediate 12: (1,6-dimethyl-2-oxo-1,2-dihydro-5-quinolinyl)acetaldehyde

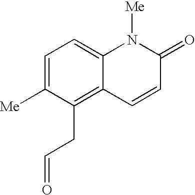

The title compound was prepared in 64% yield (2.55 g) in a similar fashion to the preparation of Intermediate 1 starting from 1,6-dimethyl-5-(2-propen-1-yl)-2(1H)-quinolinone (Intermediate 11) (3.95 g, 18.51 mmol); ¹H-NMR (400 MHz, CDCl₃) δ: 9.77 (t, 1H), 7.79 (d, 1H), 7.46 (d, 1H), 7.28 (d, 1H), 6.76 (d, 1H), 4.08 (d, 2H), 3.74 (s, 3H), 2.42 (s, 3H).

### Intermediate 13: 5-((E/Z)-2-{[2-(dimethylamino)ethyl]oxy}ethenyl)-6-methyl-2(1H)-quinolinone

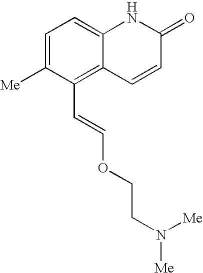

To a solution of 5-bromo-6-methyl-2(1H)-quinolinone (Intermediate 9) (1.5 g, 6.3 mmol) in DMF (50 ml) were added palladium (II) acetate (566 mg, 2.52 mmol), triphenylphosphine (661 mg, 2.52 mmol), 2-(ethenyloxy)-N,N-dimethylethanamine (5.8 g, 50.4 mmol) and TEA (7.04 ml, 50.4 mmol) and the mixture was heated at 100° C. After 2 hours further palladium (II) acetate (283 mg, 1.26 mmol), triphenylphosphine (331 mg, 1.26 mmol), 2-(ethenyloxy)-N,N-dimethylethanamine (2.9 g, 25.2 mmol) and TEA (3.52 ml, 25.2 mmol) were added and the reaction was stirred overnight at 100° C. The reaction was still not complete, so further palladium (II) acetate (142 mg, 0.63 mmol), triphenylphosphine (166 mg, 0.63 mmol), 2-(ethenyloxy)-N,N-dimethylethanamine (1.45 g, 12.6 mmol) and TEA (1.76 ml, 12.62 mmol) were added and the reaction was stirred for an additional 6 hours at 100° C. The reaction was cooled and quenched with water and extracted with DCM. The combined organic layers were dried (Na₂SO₄) and concentrated in vacuo. The crude product was purified by flash chromatography on silica gel, eluting with a gradient of methanol in ethyl acetate (5 to 10%), to afford the title compound (685 mg, 40%); (ES) m/z: 273.1 [MH⁺]; C₁₆H₂₀N₂O₂ requires 272.35.

### Intermediate 14: (6-methyl-2-oxo-1,2-dihydro-5-quinolinyl)acetaldehyde

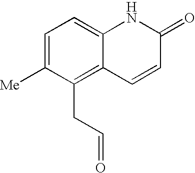

To a solution of 5-((E/Z)-2-{[2-(dimethylamino)ethyl]oxy}ethenyl)-6-methyl-2(1H)-quinolinone (Intermediate 13) (685 mg, 2.52 mmol) in DCM/pentane (50 ml/12.5 ml) was added a mixture of H₂SO₄ (96%)/H₂O (12.5 ml/50 ml). The reaction mixture was stirred at room temperature for 4 hours before neutralising with NaHCO₃ (sat. solution) and K₂CO₃. The mixture was extracted with DCM, and the combined organic layers were dried (Na₂SO₄) and concentrated in vacuo to give the title compound (442 mg) which was used in the next step without any further purification; ¹H-NMR (400 MHz, CDCl₃) δ: 12.3 (br. s., 1H) 9.77 (s, 1H), 7.95 (d, 1H), 7.43 (d, 1H), 7.35 (d, 1H), 6.77 (d, 1H), 4.08 (d, 2H), 2.43 (s, 3H).

### Intermediate 15: N-[4-chloro-3-(methyloxy)phenyl]-2,2-dimethylpropanamide

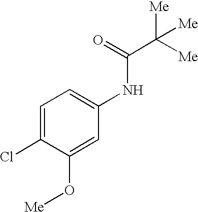

Pivaloyl chloride (7.02 ml, 57 mmol) was added over a period of 20 minutes to a solution of 4-chloro-3-(methyloxy)aniline (commercially available) (9.0 g, 57 mmol) and TEA (8.74 ml, 63 mmol) in DCM (60 ml) at ice-bath temperature. The temperature was allowed to warm to room temperature and after 2.5 hours the reaction mixture was quenched with water. The mixture was extracted with DCM and the organic layers combined, dried (Na₂SO₄) and concentrated in vacuo. The crude solid was triturated with diethyl ether to give the title compound (11.0 g, 80%); (ES) m/z: 242.1 [MH⁺]; C₁₂H₁₆ClNO₂ requires 241.72.

### Intermediate 16: N-[4-chloro-2-formyl-3-(methyloxy)phenyl]-2,2 Dimethylpropanamide

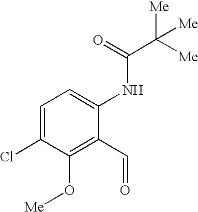

Butyllithium (71 ml of a 1.6 M sol in THF, 114 mmol) was slowly added to a solution of N-[4-chloro-3-(methyloxy)phenyl]-2,2-dimethylpropanamide (Intermediate 15) (11.0 g, 45.6 mmol) in THF (50 ml) at 0° C. The reaction was stirred at 0° C. for 2 hours and then DMF (8.8 ml, 114 mmol) was added. The temperature was raised to room temperature and stirring continued for a further 16 hours. The reaction was quenched with saturated aqueous NH₄Cl and extracted with ethyl acetate. The organic layers were combined, dried (Na₂SO₄) and concentrated in vacuo. This crude product was used in the next step without any further purification; ¹H-NMR (400 MHz, CDCl₃) δ: 11.6 (br. s., 1H), 10.35 (s, 1H), 8.50 (d, 1H), 7.52 (d, 1H), 3.95 (s, 3H), 1.30 (s, 9H).

### Intermediate 17: ethyl (2E/Z)-3-[3-chloro-6-[(2,2-dimethylpropanoyl)amino]-2-(methyloxy)phenyl]-2-propenoate

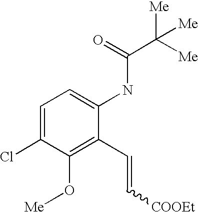

(Carbethoxymethylene)triphenyl-phosphorane (commercially available) (17.7 g, 50.9 mmol) was added to a solution of N-[4-chloro-2-formyl-3-(methyloxy)phenyl]-2,2 dimethylpropanamide (Intermediate 16) (13.7 g, 50.9 mmol) in toluene (60 ml) at room temperature. The reaction mixture was stirred for 5 hours, solvent was removed under reduced pressure and the crude product was purified by flash chromatography on silica gel, eluting with ethyl acetate/cyclohexane (3/7) to afford the title compound (13.9 g, 90%); (ES) m/z: 340.1 [MH⁺]; C₁₇H₂₂ClNO₄ requires 339.82.

### Intermediate 18: 6-chloro-5-(methyloxy)-2(1H)-quinolinone

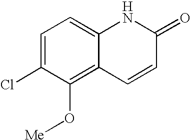

To a solution of ethyl (2E/Z)-3-[3-chloro-6-[(2,2-dimethylpropanoyl)amino]-2-(methyloxy)phenyl]-2-propenoate (Intermediate 17) (11.3 g, 33.3 mmol) in ethanol (50 ml) was added aqueous hydrochloric acid (210 ml of a 10% solution in H₂O) and the mixture was heated at reflux for 20 hours. The ethanol was removed under reduced pressure and the title product was filtered-off and washed with diethyl ether (4.42 g, 63%); (ES) m/z: 210.1 [MH⁺]; C₁₀H₈ClNO₂ requires 209.63.

### Intermediate 19: 6-chloro-5-hydroxy-2(1H)-quinolinone

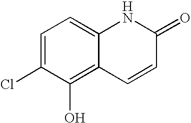

Hydrogen bromide (48% in H₂O, 60 ml) was slowly added to 6-chloro-5-(methyloxy)-2(1H)-quinolinone (Intermediate 18) (2.0 g, 9.6 mmol) and the mixture was heated at 130° C. for 2.5 hours. The solvent was removed under reduced pressure to give a solid that was washed with water and diethyl ether to give the title compound (2.19 g, 100%); (ES) m/z: 196.0 [MH⁺]; C₉H₆ClNO₂ requires 195.60.

### Intermediate 20: 6-chloro-2-oxo-1,2-dihydro-5-quinolinyl trifluoromethanesulfonate

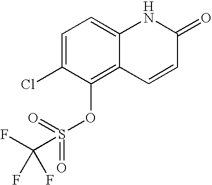

1,1,1-Trifluoro-N-phenyl-N-[(trifluoromethyl)sulfonyl]methanesulfonamide (5.21 g, 14.6 mmol) was added portion-wise to a stirred suspension of 6-chloro-5-hydroxy-2(1H)-quinolinone (Intermediate 19) (2.19 g, 11.2 mmol) in CH₃CN (60 ml) and triethylamine (4.7 ml, 33.7 mmol) at 0° C. The reaction mixture was stirred for 7 hours at room temperature then quenched with water and extracted with DCM. The combined organic layers were dried (Na₂SO₄) and concentrated in vacuo. The crude product was purified by trituration with diethyl ether to afford the title compound (2.69 g, 73%); (ES) m/z: 328.2 [MH⁺]; C₁₀H₅CIF₃NO₄S requires 327.67.

### Intermediate 21: 6-chloro-5-(2-propen-1-yl)-2(1H)-quinolinone

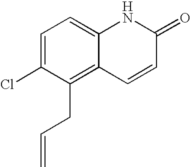

The title compound was prepared in 71% yield (952 mg) in a similar fashion to the preparation of Intermediate 11 starting from 6-chloro-2-oxo-1,2-dihydro-5-quinolinyl trifluoromethanesulfonate (Intermediate 20) (2.00 g, 6.12 mmol); (ES) m/z: 220.1 [MH⁺]; C₁₂H₁₀ClNO requires 219.67.

### Intermediate 22: 6-chloro-1-methyl-5-(2-propen-1-yl)-2(1H)-quinolinone

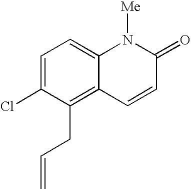

The title compound was prepared in 53% yield (469 mg) in a similar fashion to the preparation of Intermediate 2 starting from 6-chloro-5-(2-propen-1-yl)-2(1H)-quinolinone (Intermediate 21) (837 mg, 3.82 mmol); (ES) m/z: 234.1 [MH⁺]; C₁₃H₁₂ClNO requires 233.70.

### Intermediate 23: (6-chloro-1-methyl-2-oxo-1,2-dihydro-5-quinolinyl)acetaldehyde

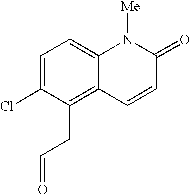

The title compound was prepared in 52% yield (220 mg) in a similar fashion to the preparation of Intermediate 1 starting from 6-chloro-1-methyl-5-(2-propen-1-yl)-2(1H)-quinolinone (Intermediate 22) (469 mg, 2.01 mmol); ¹H-NMR (400 MHz, CDCl₃) δ: 9.8 (t, 1H), 7.71 (d, 1H), 7.64 (d, 1H), 7.34 (d, 1H), 6.80 (d, 1H), 4.28 (d, 2H), 3.75 (s, 3H).

### Intermediate 24: (±)-7-bromo-3,6-dimethyl-1,1a,3,7b-tetrahydro-2H-cyclopropa[c]quinolin-2-one

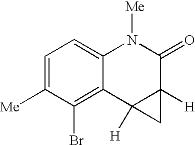

Sodium hydride (368 mg of a 60% dispersion in mineral oil, 6.7 mmol) was added to a solution of trimethylsulfonium iodide (1.43 g, 6.5 mmol) in DMSO (4 ml) and the mixture was stirred for 1 hour at room temperature. The reaction temperature was cooled to 0° C. and a solution of 5-bromo-1,6-dimethyl-2(1H)-quinolinone (Intermediate 10) in DMSO (2 ml) was added dropwise. The resulting mixture was then heated at 90° C. for 2 days. The reaction mixture was poured onto ice and extracted with ethyl acetate. The combined organics were dried (Na₂SO₄) and concentrated in vacuo. The crude product was purified by SPE cartridge (silica gel, 20 g) eluting with cyclohexane/ethylacetate (3:1) to afford the title compound (77 mg, 31%); MS: (ES) m/z: 266, 268 [MH⁺]. C₁₂H₁₂BrNO requires 266.14.

### Intermediate 25: (±)-3,6-dimethyl-7-(2-propen-1-yl)-1,1a,3,7b-tetrahydro-2H-cyclopropa[c]quinolin-2-one

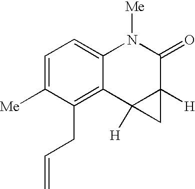

The title compound was prepared in 94% yield in a similar fashion to the preparation of Intermediate 11 starting from 7-bromo-3,6-dimethyl-1,1a,3,7b-tetrahydro-2H-cyclopropa[c]quinolin-2-one (Intermediate 24, 498 mg, 1.9 mmol); MS; (ES) m/z: 228.1 [MH⁺]. C₁₅H₁₇NO requires 227.14.

### Intermediate 26: (O)-(3,6-dimethyl-2-oxo-1a,2,3,7b-tetrahydro-1H-cyclopropa[c]quinolin-7-yl)acetaldehyde

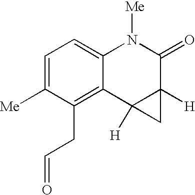

The title compound was prepared in 52% yield in a similar fashion to the preparation of Intermediate 1 starting from 3,6-dimethyl-7-(2-propen-1-yl)-1,1a,3,7b-tetrahydro-2H-cyclopropa[c]quinolin-2-one (Intermediate 25, 398 mg, 1.75 mmol); ¹H-NMR (400 MHz, CDCl₃) δ: 9.79 (s, 1H), 7.13 (d, 1H), 6.86 (d, 1H), 3.87-4.06 (m, 2H), 3.35 (s, 3H), 2.37-2.50 (m, 1H), 2.32 (s, 3H), 2.22-2.33 (m, 1H), 1.57-1.66 (m; 1H), 0.53-0.61 (m, 1H).

### Biological Assays

### a) Functional Potency—Primary Screen

The functional potency may be determined by the following GTPγS binding protocol. Cells used in the study are CHO Cells and Human Embryo Kidney (HEK293). Cells were transfected with DNA coding for human receptors as follows: HEK293_{—}5-HT_{1A}; CHO_{—}5-HT_{1B}; and CHO_{—}5-HT_{1D}. Test compounds were initially dissolved in 100% dimethyl sulfoxide to a concentration of 10 mM. Serial dilution of the test compounds in 100% dimethyl sulphoxide was carried out using a Biomek FX in 384 well assay plates, so that the final top concentration of test compound is 3 μM in the assay. Add the test compound at 1.0% total assay volume (TAV) to a solid, white, 384 well assay plate (Costar). Add 50% TAV of precoupled (for 90 mins @ RT) membranes (5 ug/well), Wheatgerm Agglutinin Polystyrene Scintillation Proximity Assay beads (RPNQ0260 Amersham International) (0.25 mg/well) in 20 mM HEPES pH 7.4, 100 mM NaCl, 3 mM MgCl₂ and 10 μM GDP. The third addition was a 20% TAV addition of either buffer, agonist format, or EC₈₀ final assay concentration (FAC) of agonist, 5HT antagonist format, prepared in assay buffer. The assay was started by the addition of 29% TAV of GTPγS 0.38 nM FAC. After all additions assay plates were incubated at RT for 2-3 hours. Assay plates were counted on a Viewlux, 613/55 filter for 5 mins. Assay plates were read between 2-6 hours after the final addition.

Using assay a), all supporting compounds 1 to 36 gave an fpKi against 5-HT_{1A} of greater than 7.7.

Using assay a), supporting compounds 1-11, 13-16, and 20-35 gave an fpKi against 5-HT_{1A} of greater than 8 and an fpKi against 5-HT_{1B} of less than or equal to 7.

Using assay a), supporting compounds 1, 2, 6-11, 13-15, 17-20, 26-29, 31, 32 and 34 gave an fpKi against 5-HT_{1A} of greater than or equal to 8.5 and an fpKi against 5-HT_{1B} of less than or equal to 7.

### b) Receptor Affinity

The affinities of the compounds of the invention for the 5-HT_{1A}, 5-HT_{1B} and 5-HT_{1D} receptors may be determined by the following assay.

Homogenise chinese hamster ovary (CHO) cells expressing 5-HT_{1A} receptors (4×10⁷ cells/ml) in Tris buffer and store in 1 ml aliquots. Homogenise CHO cells expressing 5-HT_{1B} receptors (4×10⁷ cells/ml) in Tris buffer and store in 1.5 ml aliquots. Homogenise CHO cells expressing 5-HT_{1D} receptors (1×10⁸/ml) in Tris buffer and stored in 1 ml aliquots. The binding assays are carried out in a total volume of 500 μl. For each compound to be tested make up seven solutions ranging in concentration from 0.3 mM to 0.3 nM (100× final concentrations). Dispense 5 μl of solution containing the test compound per well and add 100 μl of radioligand at 5× final desired assay concentration, i.e. [³H]-5-HT 15 nM (final assay concentration: 3 nM) in Tris Mg HCl buffer (pH 7.7) for 5-HT_{1B/1D} receptors and [³H]WAY100635 2.5 nM (final assay concentration: 0.5 nM) in Tris Mg HCl buffer (pH 7.7) containing 150 μM GPP(NH)ₚ (final assay concentration: 30 μM) for 5-HT_{1A} receptors. Add 400 μl/well of a cell membrane suspension in Tris Mg HCl buffer (pH 7.7) to make a total volume of 505 μl. Incubate at 37° C. for 45 minutes. Determine non-specific binding using 0.01 mM 5-HT for 5-HT_{1B/1D} receptors and 0.01 mM WAY100635 for 5-HT_{1A} receptors. Terminate incubation by rapid filtration using a Packard Filtermate. Measure radioactivity using Topcount scintillation counting. Calculate pKi values from the IC₅₀ generated by an iterative least squares curve fitting programme.

### c) Filtration [³H]Citalopram Binding Assay for Human SERT

The affinity of the compounds to bind the re-uptake site of serotonin transporter (SERT) may be assessed using [³H]citalopram binding assay performed in recombinant epithelial pig kidney cells stably transfected with human SERT (hSERT/LLCPK). Grow cells in Petri dishes of 500 cm² and use for membrane preparation at 80% of confluence. Harvest cells in phosphate buffered saline (PBS) containing 5 mM EDTA and centrifuge at 900 g for 8 min at 4° C. Homogenize the pellet in 30-50 vols of assay buffer (50 mM Tris, 120 mM NaCl, 5 mM KCl, 10 μM pargyline, 0.1% ascorbate (pH=7.7)) and centrifuge at 48000 g for 20 min at 4° C. Resuspend the pellet in the same volume and after incubation at 37° C. for 20 min, centrifuge as before and finally aliquot at ˜0.2 mg protein/ml in cold assay buffer. For [³H]citalopram binding assay, add 4 μl of test compound (100 times in neat DMSO) (to define total binding) or a final concentration of 10 μM fluoxetine in DMSO (to define non-specific binding), 200 μl of [³H]citalopram at final concentration of 0.25 nM in assay buffer and 200 μl of membranes diluted in assay buffer at concentration of 2 μg/well of protein (final assay volume 400 μl). Add membranes to initiate the reaction and incubate at room temperature for 2 h. Stop the reaction by rapid filtration through GF/B 96-filterplate pre-soaked in 0.5% polyethylenimmine (PEI) using a Packard cell harvester. Wash 96-filterplate 3 times with 1 ml/well cold 0.9% NaCl solution and count the radioactivity in Packard TopCount.

### d) Scintillation Proximity Assay (SPA) for Human SERT

The compound affinity to the human SERT transporter can be also assessed by using the [3H]citalopram SPA binding assay in recombinant human SERT membranes. Membranes were prepared by homogenization of Bacmam-transduced 293-F cells, followed by one centrifugation at low speed and a resuspension in a 50 mM TRIS, 130 mM NaCl buffer (membranes can be stored at −80 C for several months). SPA binding assay is performed in a 384-well plate format (Greiner 781095). Briefly, 0.5 μL of test compound in neat DMSO is added by 50 μL of the SPA mixture, containing 2 mg/mL SPA beads (Amersham RPNQ0001), 4 μg/mL hSERT Bacmam membranes, 0.01% pluronic F-127, 2.5 nM [3H]citalopram in the assay buffer (20 mM HEPES, 145 mM NaCl, 5 mM KCl, pH 7.3). Incubation is performed at room temperature for at least 2 hours. Counts are stable and can be read up to 3 days with a Trilux instruments. Only bound radioactivity can excite bead (SPA technology).

1-25. (canceled)

26. A compound of formula (I) 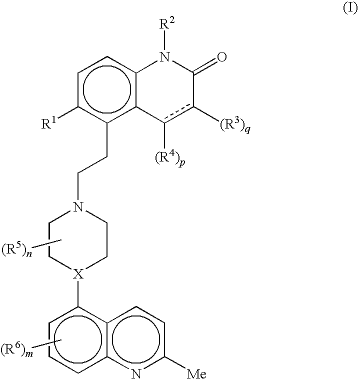
wherein
R¹ is C₁₋₆alkyl, halo or haloC₁₋₆alkyl;
R² is hydrogen or C₁₋₆alkyl; [symbol]
is a single or double bond; wherein when [symbol] is a double bond p and q are 1; and when [symbol] is a single bond, p and q are 2;
each R³ and R⁴, which may be the same or different, are hydrogen, C₁₋₆alkyl or haloC₁₋₆alkyl; or when [symbol] is a single bond, one of R³ and one of R⁴, together with their interconnecting atoms, forms a cyclopropane ring which may be substituted by one or two halo or methyl groups, which groups may be the same or different;
X is CH or N;
when present each R⁵, which may be the same or different, is C₁₋₆alkyl or halo; or two R⁵ groups may join to form a bridge, which bridge contains one or two atoms;
n is 0, 1, 2 or 3;
when present each R⁶ which may be the same or different, is C₁₋₆alkyl or halo; and
m is 0, 1, 2 or 3.

27. The compound according to claim 26, wherein
R¹ is C₁₋₆alkyl, halo or haloC₁₋₆alkyl;
R² is hydrogen or C₁₋₆alkyl; [symbol]
is a single or double bond;
each R³ and R⁴, which may be the same or different, are hydrogen, C₁₋₆alkyl or haloC₁₋₆alkyl; wherein
i) when [symbol] is a double bond p and q are 1, and
ii) when [symbol] is a single bond, p and q are 2, and one of R³ and one of R⁴, together with their interconnecting atoms, forms a cyclopropane ring which may be substituted by one or two halo or methyl groups, which groups may be the same or different;
X is CH or N;
when present each R⁵, which may be the same or different, is C₁₋₆alkyl or halo; or two R⁵ groups may join to form a bridge, which bridge contains one or two atoms;
n is 0, 1, 2 or 3;
when present each R⁶ which may be the same or different, is C₁₋₆alkyl or halo; and
m is 0, 1, 2 or 3.

28. The compound according to claim 26, wherein R¹ is C₁₋₆alkyl.

29. The compound according to claim 26, wherein R² is hydrogen or C₁₋₆alkyl.

30. The compound according to claim 26, wherein [symbol] is a single bond and each R³ and each R⁴ are hydrogen.

31. The compound according to claim 26, wherein n is 0, 1 or 2.

32. The compound according to claim 26, wherein, when present, each R⁵ is C₁₋₆alkyl.

33. The compound according to claim 26, wherein, when two R⁵ groups join to form a bridge, the bridge contains two carbon atoms and the bridge is attached to non-adjacent carbon atoms.

34. The compound according to claim 26, wherein m is 0 or 1.

35. The compound according to claim 26, wherein, when present, each R⁶ is attached to the 7-position of the quinoline ring.

36. The compound according to claim 26, wherein, the compound of formula (I) is selected from the group consisting of:
6-methyl-5-{2-[4-(2-methylquinolin-5-yl)piperazin-1-yl]ethyl}-3,4-dihydroquinolin-2(1H)-one;
5-{2-[(2S)-4-(7-fluoro-2-methylquinolin-5-yl)-2-methylpiperazin-1-yl]ethyl}-6-methyl-3,4-dihydroquinolin-2(1H)-one;
1,6-dimethyl-5-{2-[4-(2-methylquinolin-5-yl)piperazin-1-yl]ethyl}-3,4-dihydroquinolin-2(1H)-one;
6-methyl-5-{2-[4-(2-methyl-5-quinolinyl)-1-piperazinyl]ethyl}-2(1H)-quinolinone;
5-{2-[4-(7-fluoro-2-methyl-5-quinolinyl)-1-piperazinyl]ethyl}-6-methyl-2(1H)-quinolinone;
6-methyl-5-{2-[4-(2-methyl-5-quinolinyl)-1-piperidinyl]ethyl}-2(1H)-quinolinone;
5-{2-[4-(7-fluoro-2-methyl-5-quinolinyl)-1-piperazinyl]ethyl}-1,6-dimethyl-2(1H)-quinolinone;
1,6-dimethyl-5-{2-[(2S)-2-methyl-4-(2-methyl-5-quinolinyl)-1-piperazinyl]ethyl}-2(1H)-quinolinone;
5-{2-[(2S)-4-(7-fluoro-2-methyl-5-quinolinyl)-2-methyl-1-piperazinyl]ethyl}-1,6-dimethyl-2(1H)-quinolinone; and
3,6-dimethyl-7-{2-[(2S)-2-methyl-4-(2-methyl-5-quinolinyl)-1-piperazinyl]ethyl}-1,1a,3,7b-tetrahydro-2H-cyclopropa[c]quinolin-2-one.

37. The compound according to claim 26 which is 6-methyl-5-{2-[4-(2-methylquinolin-5-yl)piperazin-1-yl]ethyl}-3,4-dihydroquinolin-2(1H)-one.

38. A pharmaceutically acceptable salt of the compound according to claim 26.
